(19) United States
(12) Patent Application Publication
(10) Pub. No.: US 2011/0257130 A1
(43) Pub. Date: Oct. 20, 2011

(54) Compositions and Methods for Treatment of Renin-Angiotensin Aldosterone System (RAAS)- Related Disorders

(76) Inventor: Desikan Rajagopal, Columbus, OH (US)

(21) Appl. No.: 13/124,601

(22) PCT Filed: Oct. 16, 2009
(86) PCT No.: PCT/US09/61037

§ 371 Date: Jul. 6, 2011

(30) Foreign Application Priority Data
Oct. 17, 2008 (US) 61/196417

(51) Int. Cl. A61K 31/675; A61K 31/55; A61K 31/4535; A61K 31/445; A61K 31/4025; A61K 31/40; C07D 223/16; C07D 223/10; C07F 9/40; C07D 217/26; C07D 409/14; C07D 211/60; C07D 209/52; C07D 409/06; C07D 207/12; A61P 13/12; A61P 9/12; A61P 3/10; A61P 9/00; C12N 9/99

(52) U.S. Cl. 514/89; 435/184; 514/79; 514/91; 514/217.01; 514/217.11; 514/336; 514/354; 514/414; 514/422; 514/423; 540/542; 540/594; 540/607; 546/23; 546/142; 546/148; 546/207; 546/245; 548/413; 548/465; 548/515; 548/527; 548/533

(57) ABSTRACT

Compounds are provided which can be useful in reducing the activity of an angiotensin-converting enzyme and thus be used to treat or prevent a renin-angiotensin aldosterone system-related disorder. These compounds include lipoic acid derivatives such as prolyl lipoic acid and pipecolinyl lipoic acid, and other compounds, and these compounds are useful in treating hypertension, stroke, or other renin-angiotensin aldosterone system-related disorders in human or animal patients. Pharmaceutical compositions prepared using these compounds and methods of treatment using these compounds are also provided.

### CROSS-REFERENCE TO RELATED APPLICATIONS

The present application claims the benefit of U.S. Provisional Application Ser. No. 61/196,417, filed Oct. 17, 2008, the entire disclosure of which is incorporated herein by reference.

### FIELD OF THE INVENTION

The presently-disclosed subject matter relates to compounds and methods for treating a renin-angiotensin aldosterone system (RAAS)-related disorder. In particular, the presently-disclosed subject matter relates to compounds including prolyl lipoic acid and pipecolinyl lipoic acid amides and derivatives thereof, and other compounds that can be used to reduce angiotensin converting enzyme activity and thus be useful in the treatment of RAAS-related disorders such as hypertension, stroke, diabetes mellitus, atherosclerosis, and other cardiovascular and renal disorders.

### BACKGROUND OF THE INVENTION

In the United States and other countries, hypertension, stroke, and other disorders related to the renin-angiotensin aldosterone system (RAAS) are a major cause of widespread morbidity and mortality, causing great hardship and economic loss to millions of people throughout the world. For example, it has been estimated that nearly 600 million people worldwide are affected with hypertension, with nearly 50 million of those individuals residing in the United States. Furthermore, it has also been estimated that hypertension alone resulted in an annual expenditure of $66.4 billion dollars in the United States alone in 2007.

Despite the widespread hardship and economic consequences associated with hypertension and other related diseases, adequate and appropriate treatment of hypertension has still remained elusive for many individuals. The etiology of diseases such as hypertension is often multi-factorial and includes a variety of causes such as sedentary lifestyles, obesity, salt sensitivities, alcohol intake, vitamin D deficiency, genetic mutations, and family history. Additionally, recent evidence has strongly indicated that there is a relationship between the development and pathology of hypertension and oxidative stress and inflammation.

To date, however, and regardless of the increasing amount of evidence that oxidative stress and inflammation play a significant role in the development and pathology of hypertension, angiotensin-converting enzyme (ACE) inhibitors continue to be regarded as one of most preferred agents for the treatment of hypertension. It has been known for a number of years that ACE cleaves a C-terminal histidine-leucine dipeptide from the 10 amino acid angiotensin I (AngI) to generate angiotensin II (AngII), which is then able to mediate a variety of physiological responses. For example, in addition to the common vasoconstrictive action of AngII, which can lead to increased blood pressure and hypertension, the physiologic effects of AngII also include: ventricular remodeling of the heart, which may lead to ventricular hypertrophy and congestive heart failure; increased free radical generation in blood vessels; stimulation of the adrenal cortex to release aldosterone, which subsequently leads to increases in blood volume and increases in blood pressure; and, stimulation of the posterior pituitary to release vasopressin (also known as anti-diuretic hormone, ADH) which acts on the kidneys to increase water retention.

In light of these wide-ranging effects, the RAAS has thus been implicated extensively in the pathogenesis of hypertension, diabetes mellitus, target organ damage related to diabetes, atherosclerosis, coronary heart disease, angina, stroke, and Reynaud's disease. Accordingly, and because AngII levels, which play a central role in the RAAS, are determined by the activity of ACE, the inhibition of ACE thus has great therapeutic potential for the treatment of these disorders. Indeed, ACE inhibitors are currently approved for the treatment of high blood pressure (hypertension) and are also widely prescribed for the treatment of diabetes with target organ damage, systolic heart failure, acute coronary syndrome, and for treatment following a heart attack. The use of ACE inhibitors in these clinical conditions is considered necessary to meet the standard of care as they have been shown to improve clinical outcomes, independent of their blood pressure-lowering effects. However, prescription of ACE inhibitors for the treatment of these various disorders still largely ignores the underlying oxidative stress and inflammation that accompanies many, if not all, of these disorders. As such, individuals diagnosed with these disorders must rely on additional medications to treat the underlying inflammation and oxidative stress.

Currently, a number of anti-inflammatory agents and antioxidants are available, or are naturally-occurring, and are capable of reducing the amount of oxidative stress or inflammation in patients. In plants and animals, one such agent is alpha lipoic acid (ALA or lipoic acid). ALA, also known as thioctic acid, is a naturally-occurring 8-carbon fatty acid that is synthesized by plants and animals, including humans, and serves several important functions in the body. ALA contains two sulfur atoms that are normally found in an oxidized, disulphide form, but which can be reduced to form thiols. This feature allows forms of ALA, such as the lipomide form of ALA, to function as a cofactor for several important enzymes as well as a potent antioxidant. As a potent antioxidant, ALA can scavenge various free radicals and oxidants including hydroxyl radicals, singlet oxygens, peroxynitrite, and hypochlorous acid. Because these free radicals have been implicated in the pathophysiology of many chronic diseases, it is believed that the pharmacotherapeutic effects of ALA are largely due to its antioxidant properties. In addition to its antioxidant properties, however, ALA is also a potent anti-inflammatory reagent. ALA inhibits the activation of IKK/NF-κB signaling which plays a central role in inflammatory response. Furthermore, a recent report has demonstrated that ALA inhibited atherosclerotic lesion development, due at least in part to its anti-inflammatory effect (see, e.g., Frei B, et al. Circulation. 2008; 117:421-428).

Although certain health benefits have been attributed to the administration of exogenous ALA, ALA still continues to be largely viewed as only a nutraceutical supplement with the remainder of its underlying health benefits yet to be fully realized. Furthermore, it remains unknown as to how a molecule could be structured to obtain the maximum benefits associated with ALA and also be useful in reducing angiotensin-converting enzyme activity, since various chemical moieties which perform one function might interfere with other moieties performing a second function. Indeed, to date, ALA has failed to be effectively combined with an ACE inhibitor such that the beneficial properties of ALA and those of ACE inhibitors could be combined into one compound that is capable of exhibiting a variety of multi-functional therapeutic effects by targeting multiple diseases and their related pathways with minimal toxicity.

Accordingly, a compound combining the properties of lipoic acid and ACE inhibitors would be highly desirable and potentially very beneficial in treating a variety of disorders related to the action of the RAAS, especially those where underlying inflammation and oxidative stress accompany the disorder.

### SUMMARY OF THE INVENTION

It is thus an object of the present invention to provide compounds and methods for treating renin-angiotensin aldosterone system (RAAS)—related disorders that can provide the beneficial properties of lipoic acid yet not interfere with the function of the compound to also inhibit angiotensin-converting enzyme (ACE) and derive the benefits therein.

It is also an object of the present invention to provide methods for reducing ACE activity wherein ACE is contacted with an effective amount of a compound of the present invention.

It is still a further object of the present invention to provide methods for treating RAAS-related disorders, such as hypertension, stroke, diabetes mellitus, target organ damage related to diabetes mellitus, atherosclerosis, coronary heart disease, angina, renal disorders, or Reynaud's disease, wherein a subject in need of treatment is administered an effective amount of a compound of the present invention to thereby treat the RAAS-related disorder.

These and other objects are provided by the present invention which comprises compounds that include the beneficial properties of ACE inhibitors with those of lipoic acid. In a preferred embodiment of the present invention, compounds are provided having the general Formula (I) as follows:

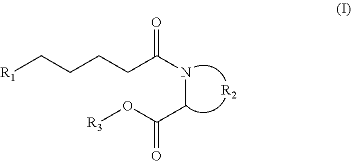

wherein:

R₁ is selected from the group consisting of

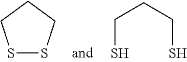

R₂ completes a 5- or 6-membered non-aromatic heterocyclic ring structure; and

R₃ is selected from the group consisting of CH₃ and H.

In another preferred embodiment of the invention, compounds are provided having the general Formula (X) as follows:

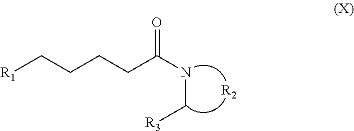

wherein:

R₁ is selected from the group consisting of:

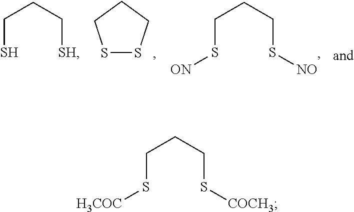

R₂ completes a five- or six-membered non-aromatic heterocyclic ring structure optionally containing a hydroxyl substituent, or an eight- or ten-membered bicyclic aromatic or non-aromatic heterocyclic ring structure; and

R₃ is selected from the group consisting of:

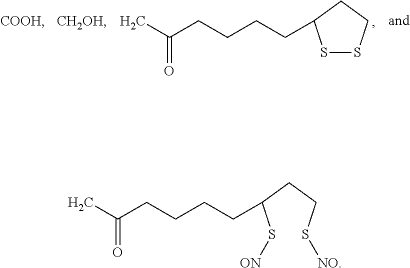

In yet another preferred embodiment of the present invention, compounds are provided having a general Formula (XI):

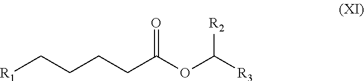

wherein:

R₁ is selected from the group consisting of:

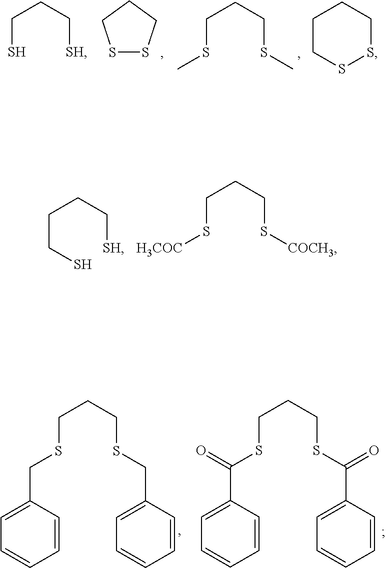

R₂ is selected from the group consisting of:
(CH₂)₂CH₃, (CH₂)₃NH₂, (CH₂)₃OH, (CH₂)₄COOH,

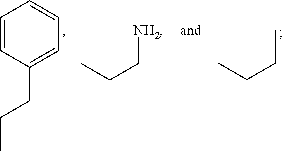

and

R₃ is selected from the group consisting of:

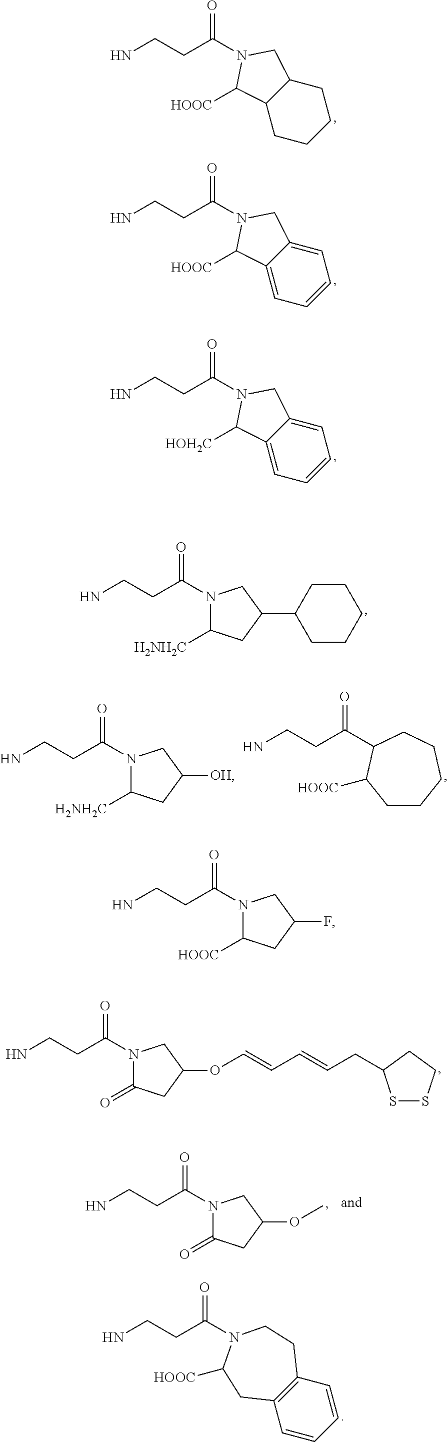

In still another preferred embodiment of the present invention, compounds are provided having a general Formula (XII) as follows:

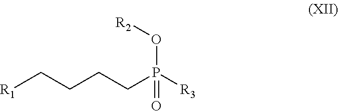

wherein:

R₁ is selected from the group consisting of:

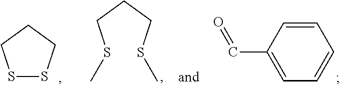

R₂ is selected from the group consisting of:

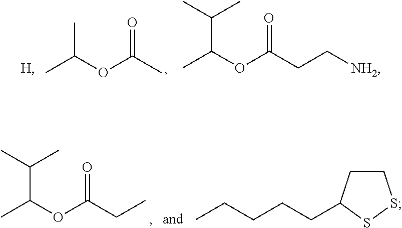

and

R₃ is selected from the group consisting of:

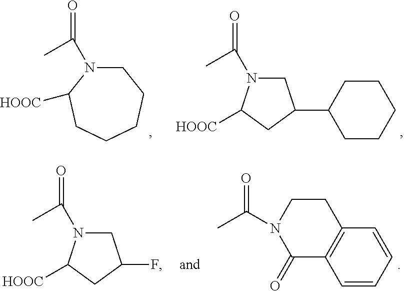

In other preferred embodiments of the present invention, compounds are provided having a general Formula (XIII) as follows:

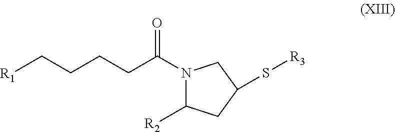

wherein:

R₁ is selected from the group consisting of:

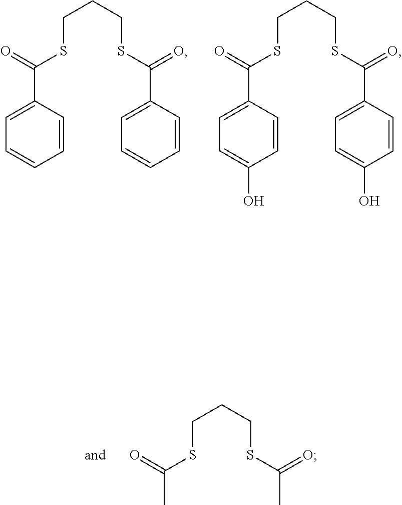

R₂ is selected from the group consisting of:
COOH,

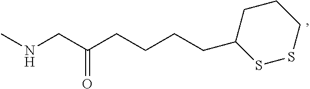

### and CH₂OCH₃; and

R₃ is selected from the group consisting of: H, CH₃, and COCH₃.

In certain embodiments of the present invention, compounds are provided that have the general Formula (XIV) as follows:

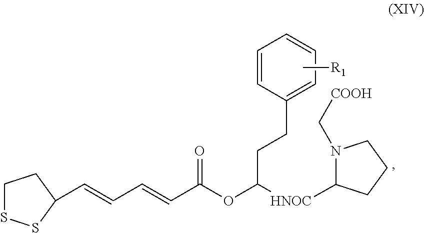

wherein R₁ is selected from the group consisting of OH, NO₂, NH₂, and OCH₃.

In other embodiments of the present invention, a compound is provided that has the general Formula (XV) as follows:

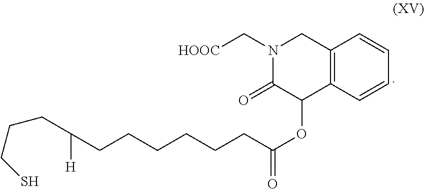

In further embodiments of the present invention, a compound is provided that has the general Formula (XVI):

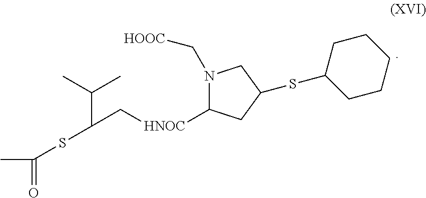

In still further embodiments of the present invention, compounds are provided that have the general Formula (XVII):

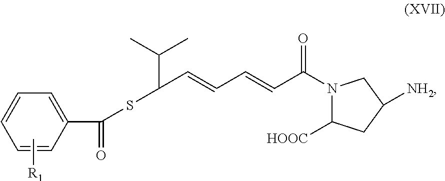

wherein R₁ is selected from the group consisting of OH, NO₂, OCH₃, and NH₂.

In yet another embodiment of the present invention, compounds are provided that have the general Formula (XVIII):

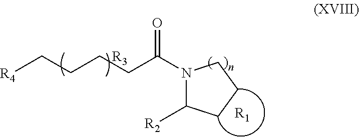

wherein R₁ is an optional ring that can be selected from the group consisting of a cyclopentyl ring, a cyclohexyl ring, a phenyl ring, and a substituted phenyl ring where the substituents on the phenyl ring are selected from the group consisting of OH, OCH₃, NH₂, NO₂, COOH;

wherein R₂ is selected from the group consisting of a free carboxyl, methyl or ethyl ester, a primary alcohol, and a primary amine group;

wherein n is an integer from 1 to 3, and the nitrogen-containing ring is a substituted or unsubstituted ring selected from the group consisting of a pyrrolidine ring, a piperidine ring, and a saturated azepine ring;

wherein R₃ is an alkyl group that includes from 1 to 3 carbon atoms, and which can include one or more conjugated or unconjugated double bonds; and

wherein R₄ is a substituted or unsubstituted 5- or 6-membered disulphide moiety or a 5- or 6-membered dithiol moiety selected from the group consisting of

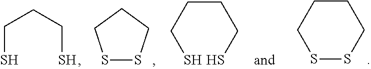

In addition, the invention provides pharmaceutical compositions made from said compounds and methods for treating diseases such as hypertension, stroke, and other disorders that make use of an effective amount of said compounds, as further described in the detailed description of the invention provided hereinbelow.

### BRIEF DESCRIPTION OF THE DRAWINGS

FIG. 1 is a schematic representation of an exemplary synthesis of L-prolyl methyl ester lipoic acid (Formula (II)) from L-proline methyl ester and (DL)-alpha lipoic acid.

FIG. 2 is a schematic representation of results from a thin layer chromatography experiment used to confirm the formation of L-prolyl methyl ester lipoic acid (Formula (II)) where point A represents lipoic acid and point B represents L-prolyl methyl ester lipoic acid (Formula (II)).

FIG. 3 shows the results of a proton nuclear magnetic resonance (NMR) spectroscopy experiment used to characterize L-prolyl methyl ester lipoic acid (Formula (II)), where peaks were assigned based on the observed chemical shift values (δ), including δ 4.448 (1H, triplet), 3.695 (3H, singlet), 3.624-3.464 (2H, multiplet), 3.184-3.098 (2H, multiplet), 2.531-2.284 (2H, multiplet), 2.150-1.895 (6H, multiplet), 1.693-1.621 (5H, multiplet), and 1.480-1.444 (4H, multiplet).

FIG. 4 is a schematic representation of an exemplary synthesis of L-prolyl lipoic acid (Formula (III)) from L-prolyl methyl ester lipoic acid (Formula (II)).

FIG. 5 is a schematic representation of results from a thin layer chromatography experiment used to confirm the formation of L-prolyl lipoic acid (Formula (III)) where point A represents L-prolyl lipoic acid (Formula (III)) and point B represents L-prolyl methyl ester lipoic acid (Formula (II)).

FIG. 6 shows the results of a proton nuclear magnetic resonance (NMR) spectroscopy experiment used to characterize L-prolyl lipoic acid (Formula (III)), where peaks were assigned based on the observed chemical shift values (δ), including δ 9.230 (1H, broad singlet), 4.533 (1H, triplet), 3.584-3.453 (2H, multiplet), 3.155-3.070 (2H, multiplet), 2.458-2.410 (2H, multiplet), 2.363-3.316 (4H, multiplet), 2.265-2.243 (2H, multiplet), 2.069-2.041 (4H, multiplet), and 2.031-1981 (4H, multiplet).

FIG. 7 is a diagram showing the chemical structure of L-prolyl lipoic acid (Formula (III)) along with its chemical name and representative characteristics.

FIG. 8 is a schematic representation of an exemplary synthesis of the dithiol derivative of L-prolyl lipoic acid, 1-(6,8-dimercaptooctanyl)pyrrolidine-2-carboxylic acid (Formula (IV)), from L-prolyl lipoic acid (Formula (III)).

FIG. 9 is a schematic representation of an exemplary synthesis of (DL)-pipecolinyl methyl ester lipoic acid (Formula (V)) from (DL)-pipecolinyl acid methyl ester and (DL)-alpha lipoic acid.

FIG. 10 is a schematic representation of results from a thin layer chromatography experiment used to confirm the formation of (DL)-pipecolinyl methyl ester lipoic acid (Formula (V)) where point A represents (DL)-pipecolinyl methyl ester lipoic acid (Formula (V)) and point B represents lipoic acid.

FIG. 11 is a schematic representation of an exemplary synthesis of (DL)-pipecolinyl lipoic acid (Formula (VI)) from (DL)-pipecolinyl methyl ester lipoic acid (Formula (V)).

FIG. 12 is a schematic representation of an exemplary synthesis of 1-(6,8-dimercaptooctanyl)piperidine-2-carboxylic acid (Formula (VII)) from (DL)-pipecolinyl lipoic acid (Formula (VI)).

FIG. 13 is a dose response curve showing the reduction in activity of angiotensin-converting enzyme in response to various concentrations of 1-(6,8-dimercaptooctanyl)pyrrolidine-2 carboxylic acid (INB; Formula (IV)).

FIG. 14 is a dose response curve showing the reduction in activity of angiotensin-converting enzyme in response to various concentrations of prolyl lipoic acid (Formula (III)).

FIG. 15 is a graph showing the ability of prolyl lipoic acid (Formula (III)) and lipoic acid to relax pre-constricted rat aortic segments.

FIG. 16 is a graph showing intra-arterial blood pressure measurements in spontaneously hypertensive stroke prone rats before and after a single 50 mg/kg dose of prolyl lipoic acid (Formula (III)).

FIG. 17 is a graph showing the mean 12-hour systolic blood pressure of spontaneously hypertensive stroke prone rats before and after a single 50 mg/kg dose of prolyl lipoic acid (Formula (III)).

FIG. 18 includes graphs showing the blood pressure of spontaneously hypertensive stroke prone rats before and after receiving an injection of prolyl lipoic acid (Formula (III)) at a concentration of 50 mg/kg.

FIG. 19 includes graphs showing the blood pressure of another group of spontaneously hypertensive stroke prone rats before and after receiving an injection of prolyl lipoic acid (Formula (III)) at a concentration of 50 mg/kg.

FIG. 20 includes graphs showing the blood pressure of spontaneously hypertensive stroke prone rats before and after receiving an injection of captopril at a concentration of 100 mg/kg.

FIG. 21 is a graph showing the extent of relaxation as a percentage of phenylephrine-induced pre-constriction in aortic segments from spontaneously hypertensive stroke prone rats treated with prolyl lipoic acid (Formula (III); PLA) at doses of 50 mg/kg or from 5 untreated spontaneously hypertensive stroke prone rats (Placebo), where the aortic segments were pre-contracted with 0.3 μM of phenylephrine and relaxed using various concentrations of acetylcholine (Ach).

FIG. 22 is a graph showing the extent of relaxation as a percentage of phenylephrine-induced pre-construction in aortic segments from spontaneously hypertensive stroke prone rats treated with prolyl lipoic acid (Formula (III); PLA) at doses of 50 mg/kg or from 5 untreated spontaneously hypertensive stroke prone rats (Placebo), where the aortic segments were pre-contracted with 0.3 μM of phenylephrine and relaxed using various concentrations of sodium nitroprusside (SNP).

FIGS. 23A-G are graphs showing expression of various inflammatory genes in aortas of spontaneously hypertensive stroke prone rats receiving daily doses of 50 mg/kg of prolyl lipoic acid (Formula (III); PLA) or a saline placebo, including graphs showing the mRNA expression of ICAM1 (FIG. 23A), IL-6 (FIG. 23B), IL-1β (FIG. 23C), MCP1 (FIG. 23D), TGF-β1 (FIG. 23E), VCAM1 (FIG. 23F), and TNF-α (FIG. 23G).

FIGS. 24A-G include graphs showing the expression of various inflammatory genes in kidneys of spontaneously hypertensive stroke prone rats receiving daily doses of 50 mg/kg of prolyl lipoic acid (Formula (III); PLA) or a saline placebo, including graphs showing the mRNA expression of ICAM1 (FIG. 24A), IL-1β (FIG. 24B), IL-6 (FIG. 24C), MCP1 (FIG. 24D), TGF-β1 (FIG. 24E), TNF-α (FIG. 24F), and VCAM1 (FIG. 24G).

FIGS. 25A-D include graphs showing the expression of various inflammatory genes in hearts of spontaneously hypertensive stroke prone rats receiving daily doses of 50 mg/kg of prolyl lipoic acid (Formula (III); PLA) or a saline placebo, including graphs showing the mRNA expression of ICAM1 (FIG. 25A), MCP1 (FIG. 25B), TGF-β1 (FIG. 25C), and TNF-α (FIG. 25D).

FIGS. 26A-G include graphs showing the expression of various inflammatory genes in brains of spontaneously hypertensive stroke prone rats receiving daily doses of 50 mg/kg of prolyl lipoic acid (Formula (III); PLA) or a saline placebo, including graphs showing the mRNA expression of ICAM1 (FIG. 26A), IL-1β (FIG. 26B), IL-6 (FIG. 26C), MCP1 (FIG. 26D), TGF-β1 (FIG. 26E), VCAM1 (FIG. 26F), and TNF-α (FIG. 26G).

FIGS. 27A and 27B includes images (FIG. 27A) and a graph (FIG. 27B) showing the effects of prolyl lipoic acid on aortic medial hypertrophy in spontaneously hypertensive stroke prone rats receiving daily doses of 50 mg/kg of prolyl lipoic acid (Formula (III); PLA) or a saline placebo.

FIG. 28 includes images showing immunostaining for CD68 in hearts of spontaneously hypertensive stroke prone rats receiving daily doses of 50 mg/kg of prolyl lipoic acid (Formula (III); PLA) or receiving a saline placebo.

FIG. 29 is a graph showing the extent of CD68 expression in histological sections of brain tissues from spontaneously hypertensive stroke prone rats receiving daily doses of 50 mg/kg of prolyl lipoic acid (Formula (III); PLA) or a saline placebo.

FIG. 30 is a graph showing the extent of CD68 expression in histological sections of kidney tissues from spontaneously hypertensive stroke prone rats receiving daily doses of 50 mg/kg of prolyl lipoic acid (Formula (III); PLA) or a saline placebo.

### DETAILED DESCRIPTION OF THE PREFERRED EMBODIMENTS

In accordance with the present invention, compounds and methods for treating a renin-angiotensin aldosterone system (RAAS)-related disorder are provided. In particular, the present invention provides compounds that include the beneficial properties of angiotensin-converting enzyme (ACE) inhibitors with those of lipoic acid. These compounds are useful for reducing ACE activity both in vitro and in vivo as well as reducing inflammation and oxidative stress. Further, these compounds are also useful for treating RAAS-related disorders. In some embodiments, the compounds can be administered as part of a pharmaceutical composition to thereby treat a RAAS-related disorder in a subject.

In one of the preferred embodiments of the present invention, compounds useful in the invention will have general Formula (I) as follows:

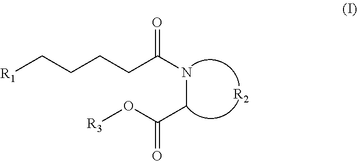

wherein:

R₁ is selected from the group consisting of

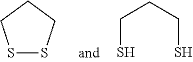

R₂ completes a 5- or 6-membered non-aromatic heterocyclic ring structure; and

R₃ is selected from the group consisting of CH₃ and H.

These compounds will include prolyl lipoic acid, prolyl methyl ester lipoic acid, pipecolinyl lipoic acid and pipecolinyl methyl ester lipoic acid. In certain embodiments, the compounds of Formula (I) may b modified so that the length of the alkyl chain in the lipoic acid moiety can be tailored to have impacts on hydrophobicity and can include alkyl chains of up to 12 carbon atoms. Specific compounds in accordance with the general Formula (I) above will include the following formulas (II) though (IX) as set forth below:

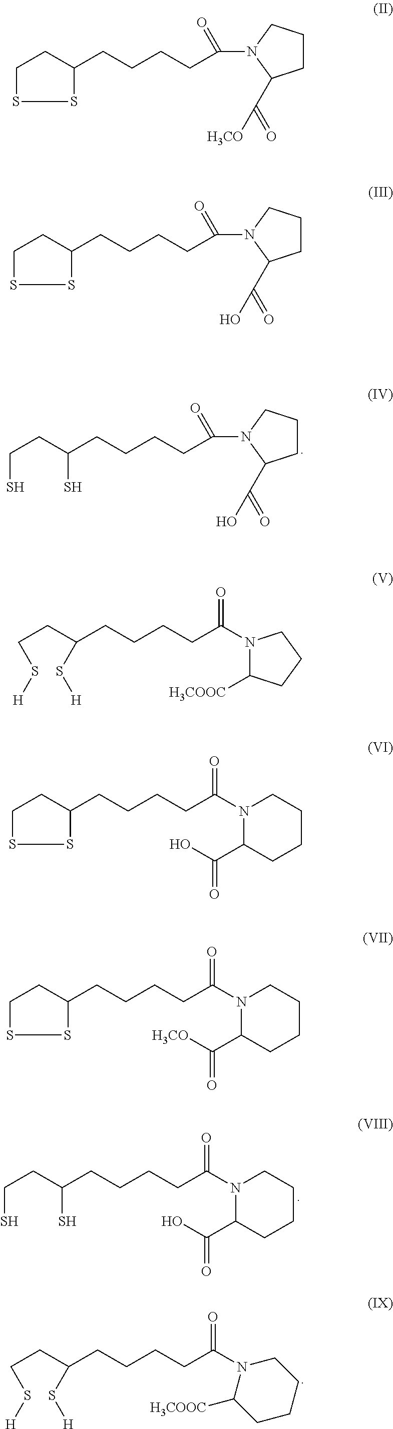

In another preferred embodiment of the invention, compounds useful in the invention will have general Formula (X) as follows:

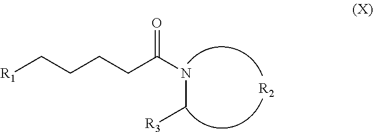

wherein:

R₁ is selected from the group consisting of:

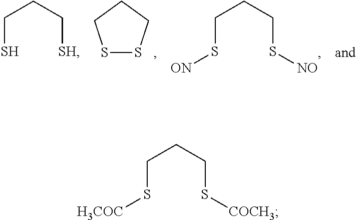

R₂ completes a five- or six-membered non-aromatic heterocyclic ring structure optionally containing a hydroxyl substituent, or an eight- or ten-membered bicyclic aromatic or non-aromatic heterocyclic ring structure; and

R₃ is selected from the group consisting of:

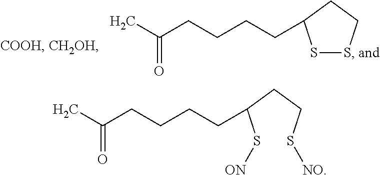

Specific compounds in accordance with the general Formula (X) above will include compounds selected from the following formulas:

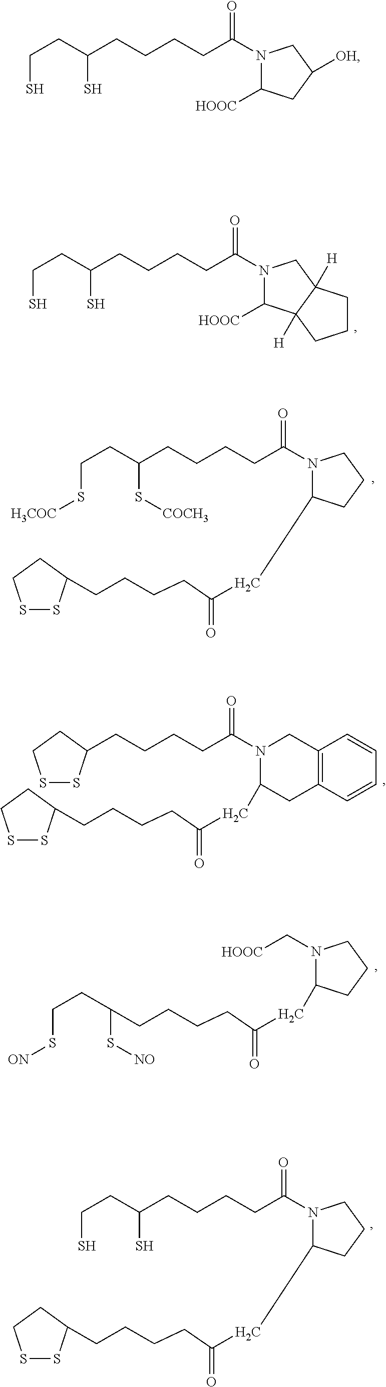

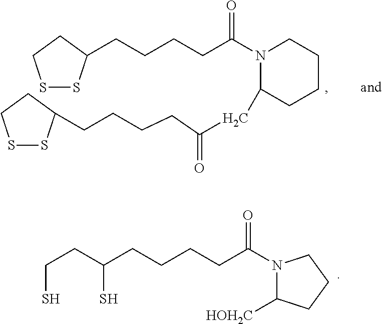

In yet another preferred embodiment of the present invention, compounds useful in the invention will have a general Formula (XI):

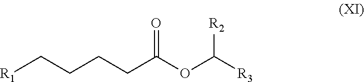

wherein:

R₁ is selected from the group consisting of:

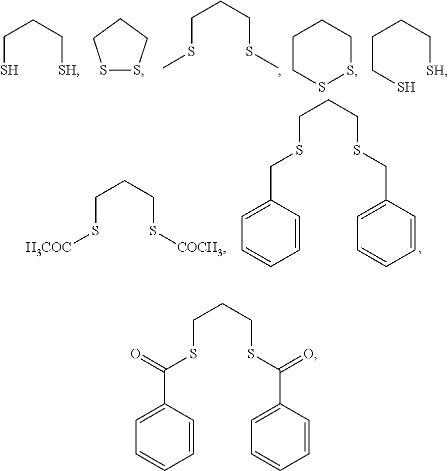

and;

R₂ is selected from the group consisting of:
(CH₂)₂CH₃, (CH₂)₃NH₂, (CH₂)₃OH, (CH₂)₄COOH,

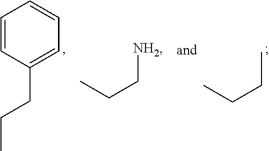

and

R₃ is selected from the group consisting of:

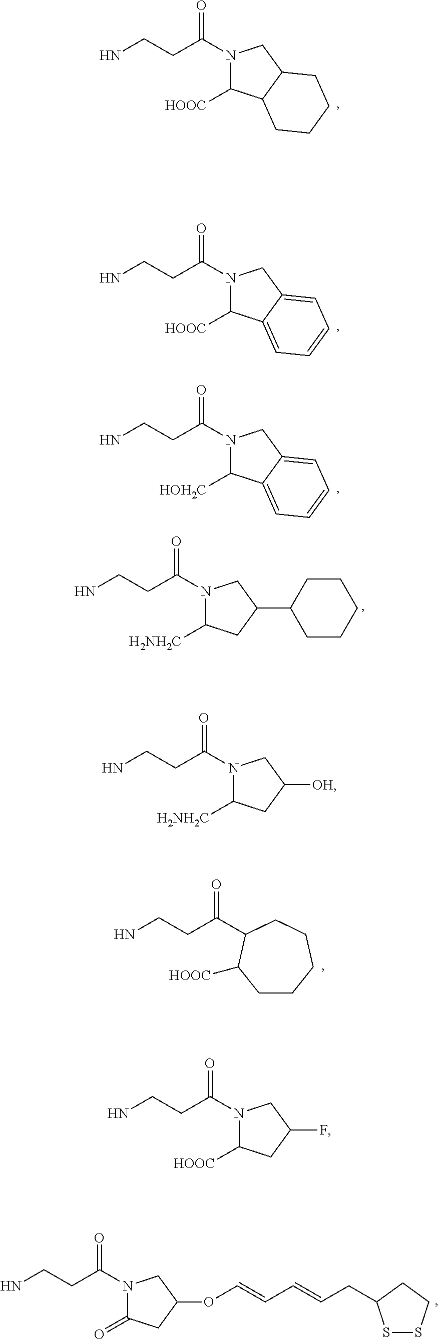

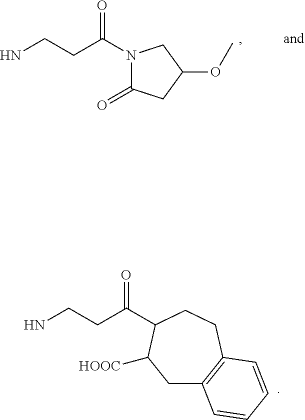

Specific compounds in accordance with the general Formula (XI) above will include compounds selected from the following formulas as set forth below:

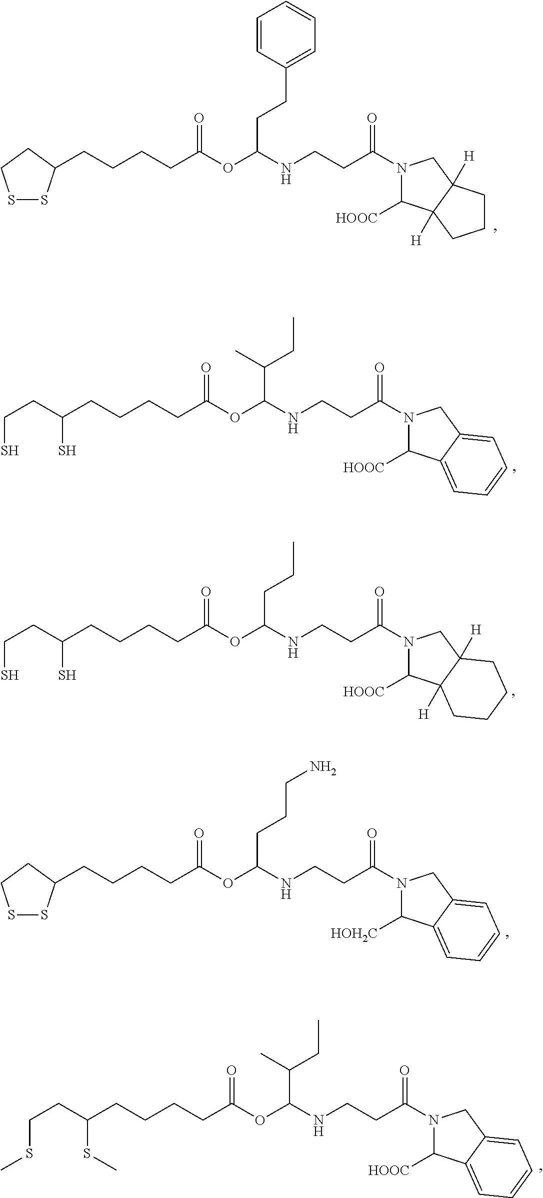

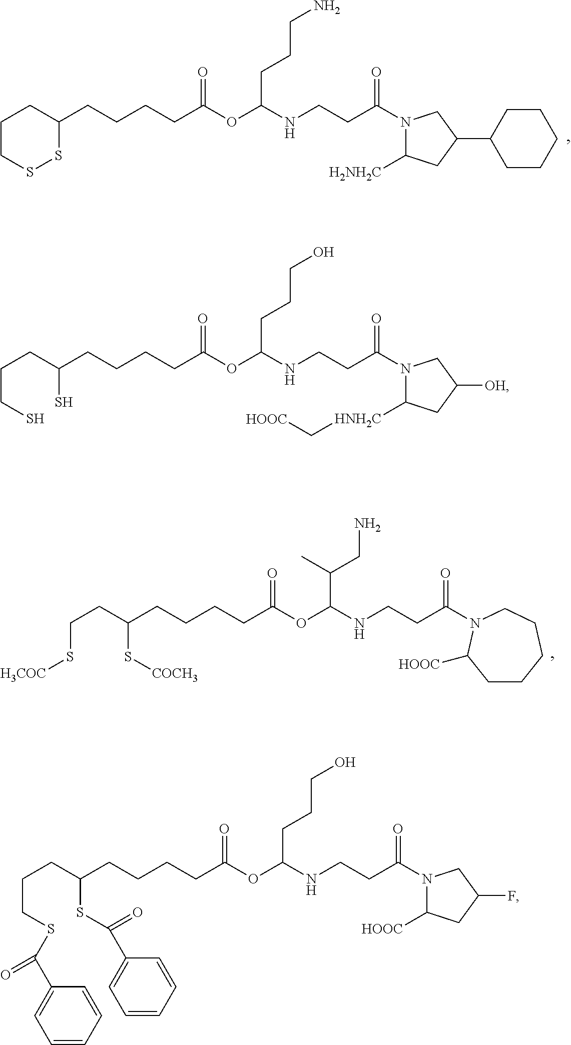

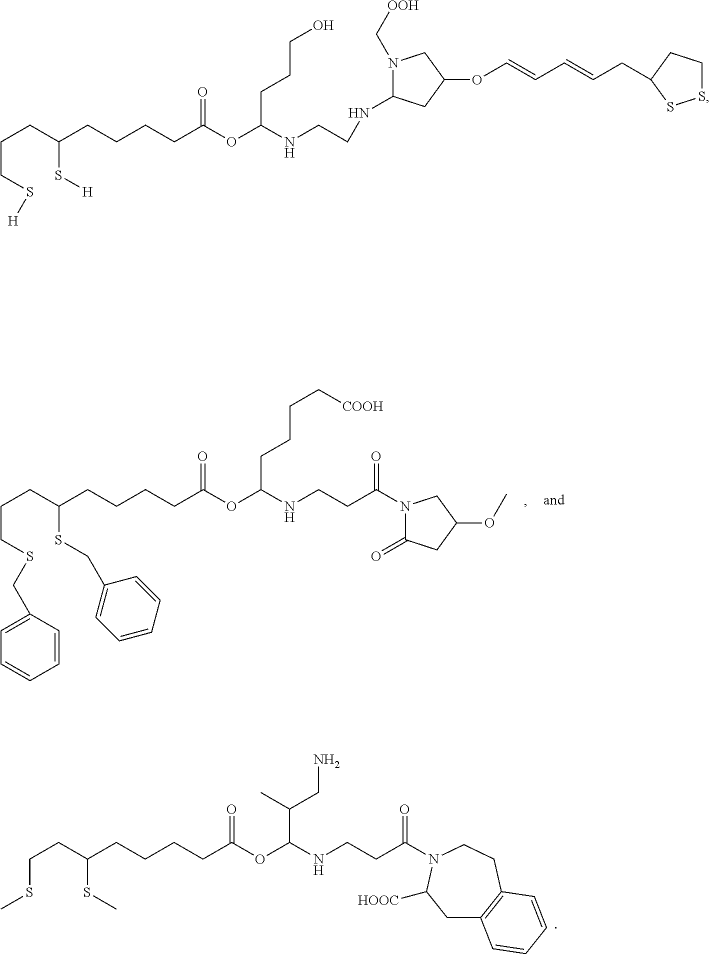

In still another preferred embodiment of the present invention, compounds useful in the invention will have a general Formula (XII) as follows:

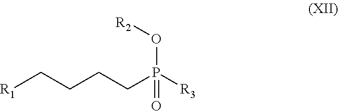

wherein:

R₁ is selected from the group consisting of:

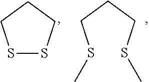

and a benzoyl group such as

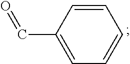

R₂ is selected from the group consisting of:

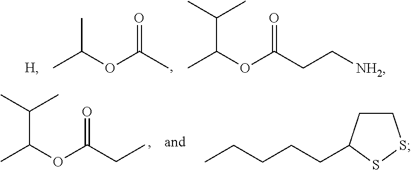

and

R₃ is selected from the group consisting of:

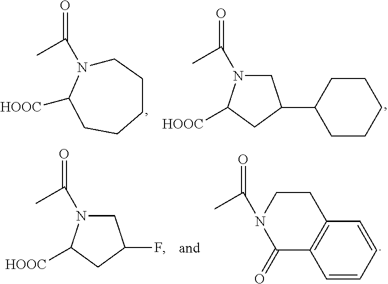

Specific compounds in accordance with the general Formula (XII) will include compounds selected from the following formulas as set forth below:

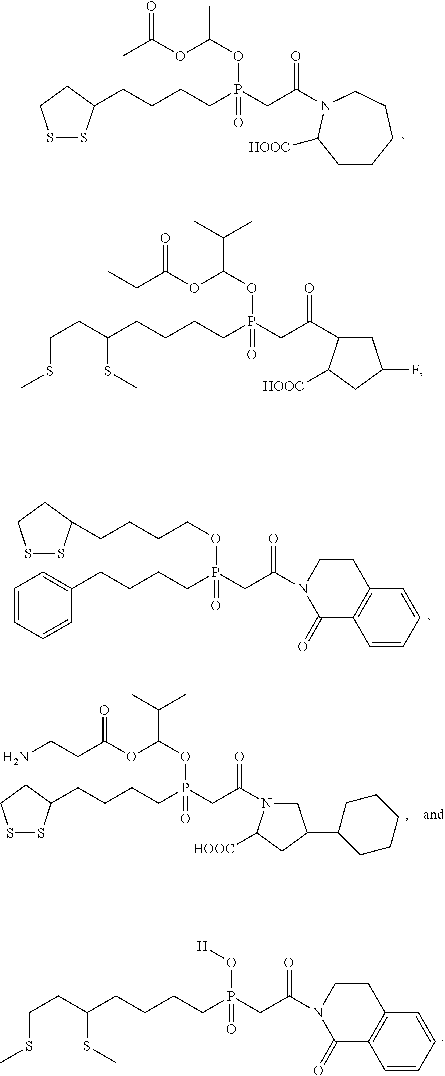

In other preferred embodiments of the present invention, compounds useful in the invention will have a general Formula (XIII) as follows:

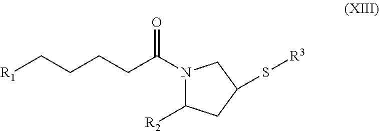

wherein:

R₁ is selected from the group consisting of:

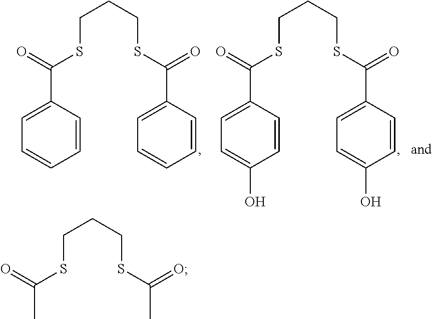

R₂ is selected from the group consisting of:
COOH,

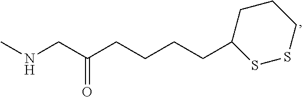

### and CH₂OCH₃; and

R₃ is selected from the group consisting of: H, CH₃, and COCH₃.

Specific compounds in accordance with the general Formula (XIII) will include compounds selected from the following formulas as set forth below:

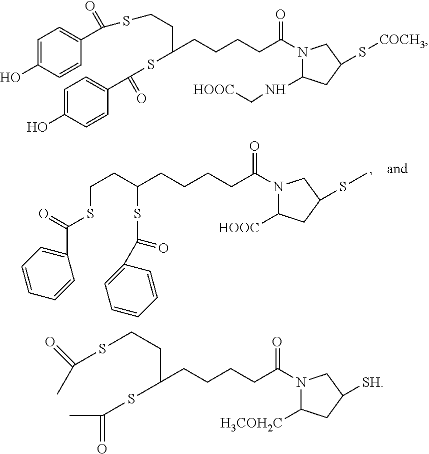

In certain embodiments of the present invention, compounds useful in the invention will have the general Formula (XIV) as follows:

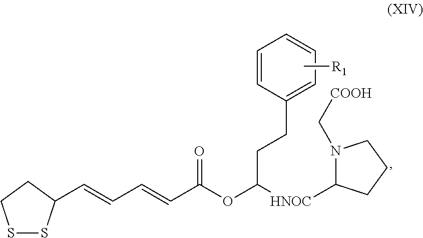

wherein R₁ is selected from the group consisting of OH, NO₂, NH₂, and OCH₃.

In other embodiments of the present invention, compounds useful in the invention will have the general Formula (XV) as follows:

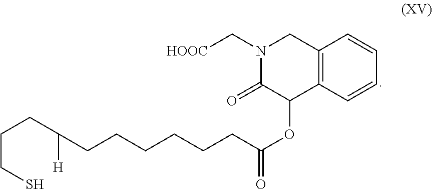

In further embodiments of the present invention, compounds useful in the invention will have the general Formula (XVI):

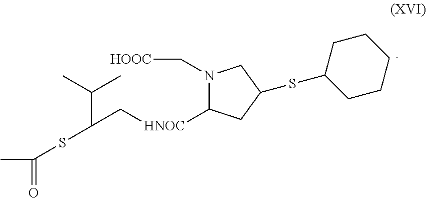

In still further embodiments of the present invention, compounds useful in the invention will have the general Formula (XVII):

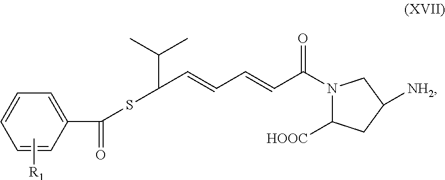

wherein R₁ is selected from the group consisting of OH, NO₂, OCH₃, and NH₂.

In yet another embodiment of the present invention, compounds are provided that have the general Formula (XVIII):

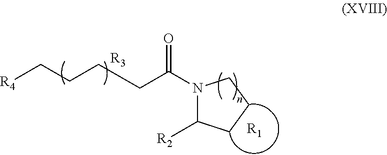

In certain embodiments of the compound of the general Formula (XVIII) described above, R₁ is optional and can be selected from a cyclopentyl ring, a cyclohexyl ring, a phenyl ring, or a substituted phenyl ring where the various substituents on the phenyl ring can include OH, OCH₃, NH₂, NO₂, COOH, or CONHR where R is methyl or ethyl. In some embodiments, the inclusion of the foregoing functional groups on the ring structure may be used for the purpose of further derivatization. In some embodiments, R₂ can be selected from a free carboxyl, methyl or ethyl ester, a primary alcohol, or a primary amine group.

In some embodiments of the compound of the general Formula (XVIII), n is an integer from 1 to 3. For example, when n is 1, the compound of Formula (XVIII) can include a pyrrolidine ring, which can additionally be substituted at the fourth carbon position with OH, SH, cyclohexyl, cyclolpentyl, amino, or methoxy groups, or which can additionally be fused with a benzene ring to from an isoindole moiety. As another example, when n is 2, the compound of Formula (XVIII) can include a piperidine ring, which can additionally be fused with a benzene ring to form a tetrahydro-isoquinoline moiety. As yet another example, when n is 3, the compound of Formula (XVIII) can include a seven-membered saturated azepine ring, which can additionally be fused with a benzene ring.

Additionally, in some embodiments of the compound of the general Formula (XVIII), R₃ can represent alkyl groups that include 1 to 3 carbon atoms, and which can further include one or more double bonds between the carbon atoms such that both conjugated and unconjugated monene, diene, and triene moieties can be incorporated in the compound of Formula (XVIII).

Further, in some embodiments of the compound of the general Formula (XVIII), R₄ is a substituted or unsubstituted 5- or 6-membered disulphide moiety or a 5- or 6-membered dithiol moiety selected from the group consisting of

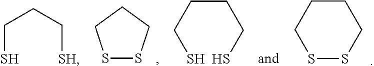

For example, when X represents a 5- or 6-membered disulphide moiety, a disulphide moiety, such as a 1,2 dithiolane group, can be incorporated into the compound of Formula (XVIII). Alternatively, when X represents a dithiol moiety, a dithiol moiety can be included in the compound of Formula (XVIII), and can be further substituted with acetyl, benzoyl, and benzyl groups.

As noted, the compounds of the present invention are useful for reducing ACE activity and are also useful for treating RAAS-related disorders. As such, various functional groups have been incorporated into the above structure to not only provide for reductions in ACE activity, but to also provide for effective treatment of RAAS-related disorders. In certain portions of the compounds above, disulfide, dithiol, acetyl-substituted dithiol, methyl-substituted dithiol, benzyl-substituted, and five- and six-membered disulfide and dithiol groups can be incorporated into the present compounds. The inclusion of a thiol group in some of the compounds can also be used for the introduction of a nitroso functional group, which may further be used as a NO donating molecule. As shown in the foregoing compounds, each of these various groups can be attached to the compounds by an extended alkyl chain that, in certain compounds, includes unsaturated double bonds such as monoenes and dienes. Furthermore, in certain portions of the compounds above, five membered pyrrolidine and six-membered pipecolinyl rings can be incorporated into the compounds, and these rings structures can further include, for example, alpha-carboxylic acid, primary alcohol, and carboxylic methyl ester substituted amide groups. In certain compounds, fluorine, thiol, substituted thiol, and amine functional groups are included in a five-membered pyrrolidine ring. In other compounds, the five-membered ring can be replaced with fused ring structures or with a seven-membered ring.

In certain embodiments of the present compounds shown above, the compounds can include a stereo-isomeric carbon atom proximal to the nitrogen atoms contained in the ring structures of the present compounds. As such, the present compounds are further inclusive of L-, D-, and D,L-isomers of the foregoing compounds.

In addition, the compounds herein are described with reference to formulas, and in these embodiments, reference to these formulas may include stereoisomers of one or more moieties of the compound. Such stereoisomers are representative of some embodiments of the compounds; however, the formulas and reference to the formulas disclosed herein are intended to encompass all active stereoisomers of the depicted compounds. Furthermore, the compounds of the presently-disclosed subject matter can, in some embodiments, contain one or more asymmetric carbon atoms and can exist in raecemic and optically active forms. All of these other forms are contemplated to be within the scope of the present invention. The compounds of the present invention can exist in stereoisomeric forms and the products obtained thus can be mixtures of the isomers.

In accordance with the invention, all of the compounds and agents of the presently-disclosed subject matter can be provided in the form of a pharmaceutically acceptable salt or solvate, as would be recognized by one skilled in the art. A salt can be formed using a suitable acid and/or a suitable base. Suitable acids that are capable of forming salts with the agents of the presently disclosed subject matter include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, or the like. Suitable bases capable of forming salts with the agents of the presently disclosed subject matter include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine, and the like), and optionally substituted ethanolamines (e.g., ethanolamine, diethanolamine, and the like).

As used herein, the term “solvate” refers to a complex or aggregate formed by one or more molecules of a solute, e.g. the compound of the present invention or a pharmaceutically-acceptable salt thereof, and one or more molecules of a solvent. Such solvates are typically crystalline solids having a substantially fixed molar ratio of solute and solvent. Representative solvents include, but are not limited to, water, methanol, ethanol, isopropanol, acetic acid, and the like. When the solvent is water, the solvate formed is a hydrate. As such, the term “pharmaceutically-acceptable salt or solvate thereof” is intended to include all permutations of salts and solvates, such as a solvate of a pharmaceutically-acceptable salt of the present compound.

In yet a further embodiment of the invention, as described further below, pharmaceutical compositions are provided which comprise the compounds as described herein with a pharmaceutically acceptable vehicle, carrier or excipient. For example, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone (POVIDONE™), hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation.

Furthermore, liquid formulations of the compounds for oral administration can be prepared in water or other aqueous vehicles, and can contain various suspending agents such as methylcellulose, alginates, tragacanth, pectin, kelgin, carrageenan, acacia, polyvinylpyrrolidone, and include solutions, emulsions, syrups, and elixirs containing, together with the active components of the composition, wetting agents, sweeteners, and coloring and flavoring agents. Various liquid and powder formulations can be prepared by conventional methods for inhalation into the lungs of the subject to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compounds can be administered by the drip method, whereby a pharmaceutical formulation containing a compound of the present invention and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically-acceptable oil base, such as an ester of a long chain fatty acid, (e.g., ethyl oleate).

In accordance with the invention, methods are provided for reducing the activity of an angiotensin-converting enzyme by contacting the enzyme with an effective amount of a compound selected from the group consisting of the following Formulas (I) and (X)-(XVIII), or pharmaceutically acceptable salts or solvates thereof. Angiotensin-converting enzyme is a peptidylcarboxypeptidase, which catalyzes the cleavage of the histidine-leucine dipeptide at the carboxy-terminus of the inactive decapeptide angiotensin I (AngI) to form AngII and is also responsible for the deactivation of bradykinase. As such, “reducing” or “the reduction of” includes, but is not limited to, reducing the cleavage of the dipeptide from the carboxy-terminus of AngI and reducing the deactivation of bradykinase. Further, it is understood that the degree of reduction need not be absolute (e.g., complete inhibition of ACE activity such that no dipeptides are cleaved from AngI), and that intermediate levels of reduction are contemplated by the present invention including, but not limited to, reductions of about 5%, about 10%, about 15%, about 20%, about 30%, about 40%, about 50%, about 60%, about 70%, about 75%, about 80%, about 85%, about 90%, about 95%, and about 99% reductions in the amount of ACE activity.

Various methods of measuring a reduction in ACE activity are known to those of ordinary skill in the art. For example, as one exemplary way to determine ACE activity in vitro, a fluorimetric procedure may be used that employs a hippury-L-histidyl-L-leucine substrate. In this assay, a reduction of ACE activity is determined by co-incubating the substrate with an ACE enzyme and a desired amount of a particular ACE inhibitor (e.g., a compound of Formula (I)), and then fluorimetrically measuring the amount of the fluorescent adduct of the enzyme-catalyzed product, L-histidyl-L-leucine. As another example of determining a reduction in ACE activity, ACE activity in vivo can be assessed by utilizing radiolabeled substrates and subsequently detecting the hydrolysis of the substrates. See, e.g., Orfanos, et al. Assays of Pulmonary Microvascular Endothelial Angiotensin-Converting Enzyme In Vivo: Comparison of Three Probes, Tox. Appl. Pharm., 1994; 124 (1): 99-111, which is incorporated herein by this reference. As yet another example of determining a reduction in ACE activity, ACE activity can be determined in a biological sample (e.g., a tissue sample, a urine sample, a saliva sample, a blood sample, a serum sample, a plasma sample, or sub-fractions thereof) obtained from a subject using commercially-available kits, as would be recognized by one of ordinary skill in the art.

As will be also recognized by those of ordinary skill in the art, in embodiments where contacting an ACE enzyme with a compound selected from the group consisting of the following Formulas (I) and (X)-(XVIII) reduces ACE activity, the optimum amount of the compound used to reduce ACE activity can vary depending on the desired degree of reduction. In certain embodiments, ACE activity is reduced by contacting ACE with a concentration of the compound in the range of about 1 to about 200 nM, such as a concentration of about 100 nM. In other embodiments, ACE activity is reduced by contacting the ACE with an effective amount of the compound by administering to a subject a dose of the compound of about 2.5 to about 400 mg per day. Of course, determination and adjustment of the amount of a compound of the present invention to be used in a particular application, as well as when and how to make such adjustments, can be ascertained using only routine experimentation.

In accordance with the invention, methods for treating an RAAS-related disorder using the above compounds are provided. In one preferred embodiment, a method for treating an RAAS-related disorder is provided that comprises administering to a subject an effective amount of a compound selected from the group consisting of the following Formulas (I) and (X)-(XVIII), or pharmaceutically acceptable salts or solvates thereof, to thereby treat the disorder in the subject.

As used herein, the terms “treatment” or “treating” relate to any treatment of a RAAS-related disorder, including but not limited to prophylactic treatment and therapeutic treatment. As such, the terms “treatment” or “treating” include, but are not limited to: preventing a RAAS-related disorder or the development of a RAAS-related disorder; inhibiting the progression of a RAAS-related disorder; arresting or preventing the further development of a RAAS-related disorder; reducing the severity of a RAAS-related disorder; ameliorating or relieving symptoms associated with a RAAS-related disorder; and causing a regression of a RAAS-related disorder or one or more of the symptoms associated with a RAAS-related disorder.

The term “renin-angiotensin aldosterone system-related disorder” or “RAAS-related disorder” is used herein to refer to disorders that are caused by, at least in part, or exacerbated by the actions of the renin-angiotensin aldosterone system. As noted herein, AngII is a central mediator of the action of the RAAS and mediates a variety of effects in subjects including: vasoconstriction, which can lead to increased blood pressure and hypertension; ventricular remodeling of the heart, which may lead to ventricular hypertrophy and congestive heart failure; increased free radical generation in blood vessels; stimulation of the adrenal cortex to release aldosterone, which subsequently leads to increased blood volume and hence an increase in blood pressure; stimulation of the posterior pituitary to release vasopressin (also known as anti-diuretic hormone, ADH) which also acts on the kidneys to increase water retention; increased inflammation and expression of various inflammatory genes, which can lead to inflammation in an affected subject; endothelial dysfunction; and vascular plaque development. In addition to the actions of AngII, the activation of the RAAS has also been implicated in, for example: reactive oxygen species development; activation and adhesion of monocytes to vascular walls; increased uptake of modified low density lipoprotein into monocytes, which creates atherogenic “foam cells;” and reduced endothelial synthesis of nitric oxide. Given these wide-ranging effects of the RAAS, and in particular AngII, the RAAS has thus been implicated in a variety of disorders including, but not limited to, hypertension, diabetes mellitus, target organ damage related diabetes mellitus, atherosclerosis, coronary heart disease, angina, stroke, and Reynaud's disease. See, e.g., Ferrario CM, Role of Angiotensin II in Cardiovascular Disease: Therapeutic Implications of More Than a Century of Research, J Renin Angiotensin Aldosterone Syst, 2006; 7: 3-14, which is incorporated herein by reference. As such, in certain embodiments, the RAAS-related disorder is selected from hypertension, diabetes mellitus, target organ damage related to diabetes mellitus, atherosclerosis, coronary heart disease, angina, stroke, renal disorders, and Reynaud's disease.

For administration of a therapeutic compound as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a compound in accordance with the methods of the present invention include, but are not limited to, systemic administration, parenteral administration (including intravascular, intramuscular, intraarterial administration), oral delivery, buccal delivery, subcutaneous administration, intraperitoneal administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

Regardless of the route of administration, the compounds of the present invention are typically administered in amount effective to achieve the desired response. As such, the term “effective amount” is used herein to refer to an amount of the therapeutic composition (e.g., a compound of Formula (I) and a pharmaceutically vehicle, carrier, or excipient) sufficient to produce a measurable biological response (e.g., a reduction in a ACE activity). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art.

In certain embodiments of the methods of treating a RAAS-related disorder disclosed herein, the compound of the present invention may be administered to a subject at a dose of about 2.5 to about 400 mg per day to achieve the desired biological response.

For additional guidance regarding formulation and dose, see U.S. Pat. Nos. 5,326,902 and 5,234,933; PCT International Publication No. WO 93/25521; Berkow, et al., (1997) The Merck Manual of Medical Information, Home ed. Merck Research Laboratories, Whitehouse Station, N.J.; Goodman, et al., (2006) Goodman & Gilman's the Pharmacological Basis of Therapeutics, 11th ed. McGraw-Hill Health Professions Division, New York; Ebadi. (1998) CRC Desk Reference of Clinical Pharmacology. CRC Press, Boca Raton, Fla.; Katzung, (2007) Basic & Clinical Pharmacology, 10th ed. Lange Medical Books/McGraw-Hill Medical Pub. Division, New York; Remington, et al., (1990) Remington's Pharmaceutical Sciences, 18th ed. Mack Pub. Co., Easton, Pa.; Speight, et al., (1997) Avery's Drug Treatment: A Guide to the Properties, Choice, Therapeutic Use and Economic Value of Drugs in Disease Management, 4th ed. Adis International, Auckland/Philadelphia; and Duch, et al., (1998) Toxicol. Lett. 100-101:255-263, each of which are incorporated herein by reference.

In certain embodiments of the presently-disclosed methods of treating a RAAS-related disorder, administering the compound of present invention to the subject reduces the activity of ACE in the subject. As noted, ACE plays an integral role in converting AngI to AngII and thus plays a role in the regulation of blood pressure. By administering a compound of the present invention to a subject though, it has been discovered that the compounds of the present invention are able to reduce the activity of ACE and thus lower the blood pressure of a subject affected by a RAAS-related disorder. As such, in certain embodiments of the therapeutic methods, administering a compound of the present invention lowers blood pressure in the subject.

In other embodiments of the therapeutic methods, administering the compound to the subject reduces an amount of expression of an inflammatory gene in a subject. As also noted herein, recent evidence has indicated that hypertension, which is mediated, in part, by increased AngII levels in a subject, is closely related to the amount of oxidative stress and inflammation in a subject. It has been discovered though that by administering a compound of the present invention to a subject affected with an RAAS-related disorder, the amount of inflammatory gene expression in the subject can be advantageously reduced, particularly in the organs and tissues of the subject most affected by the RAAS-related disorder. In certain embodiments, administering a compound of Formula (I) to a subject reduces an amount of an inflammatory gene selected from ICAM1, IL-6, IL-1β, MCP1, TGF-β1, VCAM1, TNF-α, or combinations thereof. In some embodiments, the amount of expression of an inflammatory gene is reduced in aortic, heart, or kidney tissues of a subject. In other embodiments, the amount of expression of the inflammatory genes IL-6 and ICAM1 are reduced in brain tissues of a subject.

Various methods known to those skilled in the art can be used to determine a reduction of an amount of expression of an inflammatory gene in a subject. For example, in certain embodiments, the amounts of expression of an inflammatory gene in a subject can be determined by probing for mRNA of the inflammatory gene (e.g., ICAM1, IL-6, IL-1β, MCP1, TGF-β1, VCAM1, TNF-α) in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, Calif., U.S.A.). Because the probes to which the nucleic acid molecules of the sample are bound are known, the molecules in the sample can be identified. In this regard, DNA probes for one or more of the mRNAs encoded by the inflammatory genes can be immobilized on a substrate and provided for use in practicing a method in accordance with the present invention.

With further regard to determining amounts of expression of inflammatory genes in samples, mass spectrometry and/or immunoassay devices and methods can be used to measure the peptide products of the inflammatory genes in samples, although other methods are well known to those skilled in the art as well. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Any suitable immunoassay can be utilized, for example, enzyme-linked immunoassays (ELISA), radioimmunoassays (RIAs), competitive binding assays, and the like. Specific immunological binding of the antibody to the peptide can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

The use of immobilized antibodies or fragments thereof specific for the peptides products of inflammatory genes is also contemplated by the present invention. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

Mass spectrometry (MS) analysis can be used, either alone or in combination with other methods (e.g., immunoassays), to determine the presence and/or quantity of the expression of an inflammatory gene in a subject. Exemplary MS analyses that can be used in accordance with the present invention include, but are not limited to: liquid chromatography-mass spectrometry (LC-MS); matrix-assisted laser desorption/ionization time-of-flight MS analysis (MALDI-TOF-MS), such as for example direct-spot MALDI-TOF or liquid chromatography MALDI-TOF mass spectrometry analysis; electrospray ionization MS (ESI-MS), such as for example liquid chromatography (LC) ESI-MS; and surface enhanced laser desorption/ionization time-of-flight mass spectrometry analysis (SELDI-TOF-MS). Each of these types of MS analysis can be accomplished using commercially-available spectrometers, such as for example triple quadropole mass spectrometers. Methods for utilizing MS analysis to detect the presence and quantity of peptides in biological samples are known in the art. See, e.g., U.S. Pat. Nos. 6,925,389; 6,989,100; and 6,890,763 for further guidance, each of which are incorporated herein by this reference.

Although certain embodiments of the methods disclosed herein only call for a qualitative assessment of the presence or absence of the expression of an inflammatory gene in a subject, other embodiments of the methods call for a quantitative assessment of the amount of expression of each of the inflammatory genes in the subject. Such quantitative assessments can be made, for example, using one of the above mentioned methods, as will be understood by those skilled in the art.

The skilled artisan will also understand that measuring a reduction in the amount of expression of an inflammatory gene in a subject is a statistical analysis. For example, a reduction in an amount of expression of an inflammatory gene in a subject can be compared to control level of expression, and expression of the inflammatory gene of less than or equal to the control level can be indicative of a reduction in the amount of expression, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In a specific embodiment of the present invention, a method is provided whereby hypertension is reduced by contacting an angiotension-converting enzyme with an effective amount of a compound having a formula as described above. When the method is practiced in a subject in need, the subject is preferably administered an amount of such a compound in an amount effective to reduce hypertension. As indicated above, the effective amount for any particular subject will vary based on the subject's circumstances, and would be readily determined by one of ordinary skill in the art.

In another specific embodiment of the present invention, a method for treating stroke is provided whereby a subject in need of treatment is administered an amount of a compound in accordance with the invention that is effective to treat stroke. Once again, the effective amount for any particular subject will vary based on the patient's circumstances, and would be readily determined by one of ordinary skill in the art.

Still further, as noted above, the compounds of the present invention are designed to include the beneficial properties of ACE inhibitors with those of lipoic acid. As such, it is believed that the presently-disclosed compounds will be useful as potent antioxidants, anti-inflammatory compounds, and as mitochondrial protective agents. Consequently, it is thus further contemplated that the presently-disclosed compounds can be useful for the treatment of a number of RAAS-related disorders where a reduction in ACE activity or the beneficial properties of lipoic acid are indicated.

For example, it is contemplated that the present compounds will be particularly useful in the treatment of diabetes. In this regard, it is contemplated that the compounds of the present invention will be useful for reducing oxidative stress, improving insulin signaling, treating diabetic complications that occur from overproduction of reactive oxygen and nitrogen species, and preventing the age-dependent development of hyperglycemia, hyperinsulinemia, dyslippidemia, and plasma markers of oxidative stress. Furthermore, it is also contemplated that the present compounds will be useful for preventing the mitochondrial decay that has been postulated to account for a considerable portion of the metabolic dysfunction that occurs in diabetes.

As another example, it is also contemplated that the present compounds will be useful for treating target organ damage that accompanies various RAAS-related disorders, such as hypertension, myocardial infarction, stroke, atherosclerosis, and diabetes. In this regard, it is contemplated that the present compounds will be capable of improving endothelial dysfunction by, for example, improving acetylcholine-induced endothelium dependent vasorelaxation, reducing adhesion molecules and chemokines, lowering serum triglycerides, and lowering inflammatory gene expression.

In yet a further application of the present invention, it is contemplated that the compounds as described herein will have embodiments wherein they further incorporate NO groups, and it is contemplated that the present compounds will be useful in treating angina by making NO molecules available to endothelium for vasodilation and thereby reversing or inhibiting coronary vasospasm that may occur in a subject.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The embodiments of the presently-disclosed subject matter as set forth herein are subject to modifications, and other modified embodiments within the scope of the invention will be evident to those of ordinary skill in the art after a study of the information provided in this document. The information provided in this document, and particularly the specific details of the described exemplary embodiments, is provided primarily for clearness of understanding and no unnecessary limitations are to be understood therefrom.

While the terms used in the application are believed to be well understood by one of ordinary skill in the art, definitions are set forth to facilitate explanation of the presently-disclosed subject matter. Further, unless defined otherwise, all technical and scientific terms used herein have the same meaning as commonly understood by one of ordinary skill in the art to which the present invention belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “an angiotensin-converting enzyme” includes a plurality of such enzymes, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the present invention.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Compound Synthesis Scheme

To synthesize the compound of the present invention, a general synthesis approach is utilized that involves three basic steps. In the first step, a coupling reaction is performed to connect a pyrrolidine nitrogen-containing ring structure with the carboxylic acid group of lipoic acid to form an inert amide bond. Suitable coupling reagents that may be used in this step of the procedure include: EDCI (N-(3-Dimethylaminopropyl)-N′-ethylcarbodiimide hydrochloride; DCC (dicyclohexyl carbodiimide); and CDMT (2-chloro-4,6-dimethoxy-1,3,5-triazine). Also, in this first step, the bases that are used can include dimethylaminopyridine, triethylamine; and pyridine. Suitable chlorinated organic solvents that can be used in the first step include: dichloromethane (methylenechloride), chloroform, carbon tetrachloride, dichloroethane, and tetrachloroethane.

Briefly, the first step of the reaction is carried out under a nitrogen atmosphere. A Lipoic acid-like molecule, (1 mM), an α-carboxy-pyrrolidine- or piperidine-type molecules, (1 mM), and 1 equivalent of base are first combined in a 100 mL round bottom flask. The contents of the flask are dissolved in chlorinated organic solvent (40 mL) and stirred well for 10 minutes at room temperature. The coupling reagent, (1.5 mM) is then added in portions over a period of 3 hours while the contents of the flask are simultaneously stirred. The completion of the reaction is monitored using thin layer chromatography. After overnight stirring, the solvent is then evaporated under reduced pressure using a rotary evaporator. The resultant crude product is then purified using fluorescent preparative thin layer (FPTL) chromatography with suitable chromatographic solvents such that the separation of compound is within acceptable limits. The required compound band is then scraped and the compound is extracted from the band by continuous elutions with ethylacetate. Subsequently, the solvent is evaporated to dryness and the last traces of solvent are removed under a vacuum pump. The compound is then characterized using proton nuclear magnetic resonance (NMR) spectroscopy

The second step of the reaction procedure, if desired, can then include base catalyzed hydrolysis using aqueous alcoholic potassium hydroxide (1 mM) solution or aqueous alcoholic sodium hydroxide (1 mM) solution. Briefly, in the second step of the reaction procedure, the coupled compound is de-esterified by the addition of 1M of an aqueous alcoholic alkali reagent under reflux conditions. The coupled compound (0.5 mM) is placed in a 50 mL round bottom flask that is equipped with a reflux condenser. 25 mL of 1 mM alcoholic alkali reagent is then added and set to reflux for 24 hours. Progress of the reaction is monitored by thin layer chromatography. After overnight reflux, the ethanol is then removed from the reaction mixture under reduced pressure and the removal of the ethanol is followed by the addition of water. The aqueous phase is then extracted with chlorinated organic solvent (two times) and carefully transferred to a conical flask, cooled well on crushed ice and acidified with 1 N hydrochloric acid until the pH of the solution became acidic. The aqueous phase is then extracted with chlorinated solvent, wash with brine, dried over anhydrous magnesium sulfate, and filtered. The solvent is evaporated under reduced pressure and the resulting compound is characterized using proton NMR spectroscopy, where the peaks are assigned based on the compound's chemical shift values.

The third step of the reaction procedure, if desired, can then make use of a reducing agent, such as sodium borohydride (e.g., for mild conditions), and an alcoholic medium, such as ethanol or methanol. In this third step of the reaction procedure, the disulfide portion of the molecule is converted into a dithiol moiety. Briefly, the disulphide compound is dissolved in 25 mL of ethanol and stirred well for 5 minutes. Sodium borohydride (2 equivalent) can then added to the solution by portions over a 2 hour period. Next, in order to derive the crude product, the ethanol is evaporated under reduced pressure, followed by treatment with saturated ammonium chloride. The organic compound in the aqueous phase is then extracted with chlorinated organic solvent and evaporated to dryness under reduced pressure.

The resultant mercapto derivative is then purified on column chromatography to get the compound in a purified form.

### Example 2

### Synthesis of 1-(6,8-Dimercaptooctanyl)Pyrrolidine-2-Carboxylic Acid

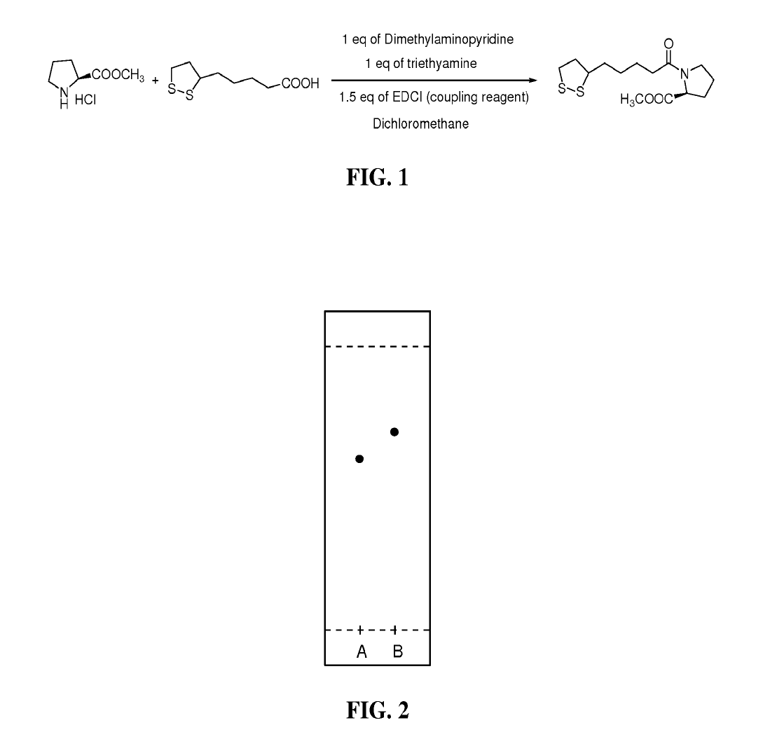

The synthesis of 1-(6,8-dimercaptooctanyl)pyrrolidine-2-carboxylic acid was performed by a three-step procedure starting from optically active L-proline methyl ester and alpha lipoic acid. L-prolyl methyl ester lipoic acid was synthesized by coupling L-proline methyl ester and lipoic acid using a suitable coupling reagent as shown in FIG. 1. Briefly, the first step of the reaction was carried out under a nitrogen atmosphere. Lipoic acid, 0.206 g (1 mM), L-proline methyl ester hydrochloride, 0.166 g (1 mM), 1 equivalent of dimethylaminopyridine (DMAP) 0.122 g (1 mM), and triethylamine 0.101 g (0.140 mL) were combined in a 100 mL round bottom flask. The contents of the flask were dissolved in methylenechloride (40 mL) and stirred well for minutes at room temperature. The coupling reagent, 1-ethyl-3-(3-dimethylaminopropyl)carbodiimide (EDCI), 0.287 g (1.5 mM) was added in portions over a period of 3 hours while the contents of the flask were simultaneously stirred. The completion of the reaction was monitored using thin layer chromatography by comparing the disappearance of starting materials and the formation of the new product, as shown in FIG. 2.

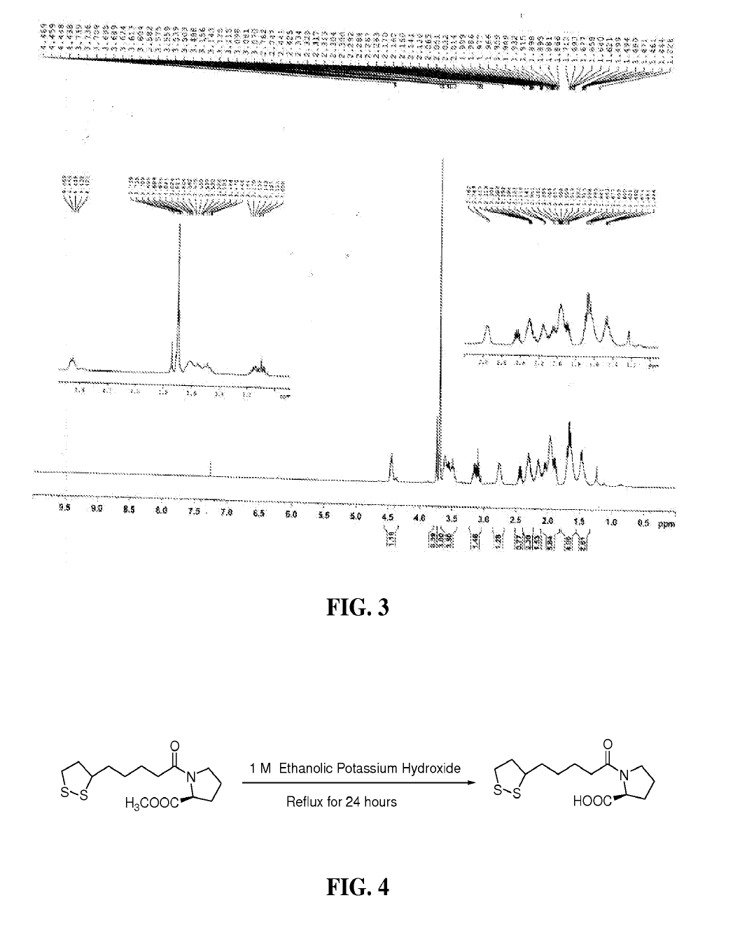

After overnight stirring, the methylenechloride was evaporated under reduced pressure using a rotary evaporator. The resultant crude product was purified using fluorescent preparative thin layer (FPTL) chromatography with a ratio of 90:5:10 ethylacetate:hexane:methanol. The required compound band was scraped and the compound was extracted from it by continuously eluting with ethylacetate (350 mL). The solvent was evaporated to dryness and the last traces of solvent were removed under a vacuum pump. A yellow semi-solid was obtained in a 62% yield. The compound was characterized using proton nuclear magnetic resonance (NMR) spectroscopy, and peaks were assigned based on the compound's chemical shift values as shown in FIG. 3. The mass of the compound was 318.1 (M+1), and, at this point in the reaction, the compound was determined to be L-prolyl methyl ester lipoic acid (Formula (II)).

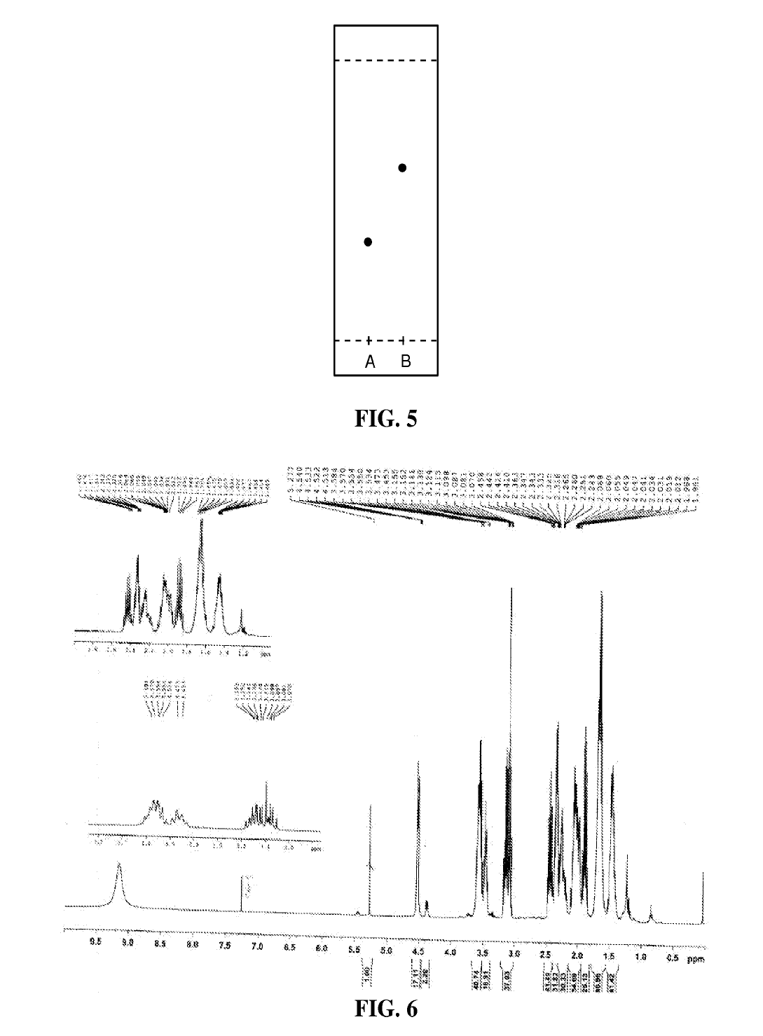

In the second step of the reaction procedure, L-prolyl lipoic acid was synthesized by the de-esterification of L-prolyl methyl ester lipoic acid in 1M ethanolic potassium hydroxide under reflux conditions as shown in FIG. 4. Similar to the first portion of the reaction procedure, this reaction step was also carried out under a nitrogen atmosphere. Briefly, L-prolyl methyl ester lipoic acid, 0.158 g (0.5 mM) was placed in a 50 mL round bottom flask that was equipped with a reflux condenser. 25 mL of 1 mM ethanolic potassium hydroxide was added and set to reflux for 24 hours. Progress of the reaction was monitored by thin layer chromatography by comparing the disappearance of starting materials and the formation of the new product as shown in FIG. 5.

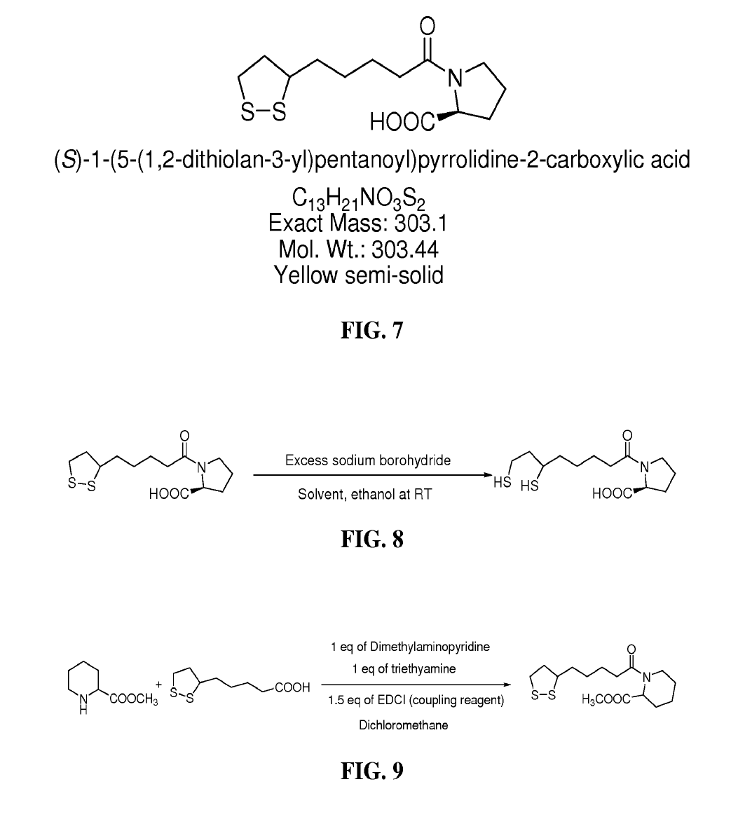

After overnight reflux, ethanol was then removed from the reaction mixture under reduced pressure and the removal of the ethanol was followed by the addition of 30 mL of water. The aqueous phase was then extracted with dichloromethane (2×25 mL) and carefully transferred to a 100 mL conical flask, cooled well on crushed ice and acidified with 1 N hydrochloric acid until the pH of the solution became acidic. The aqueous phase was extracted with dichloromethane (2×50 mL), washed with brine, dried over anhydrous magnesium sulfate, and filtered. The solvent was evaporated under reduced pressure, and a yellow colored semi-solid was obtained in a 75% yield. The resulting compound was characterized using proton NMR spectroscopy, and peaks were assigned based on the compound's chemical shift values as shown in FIG. 6. The mass of the compound was 326.1 (M+23), 607.3 (Dimer), where M+23 indicates a sodium adduct (Na MW=23), and, at this point in the reaction, the compound was determined to be L-prolyl lipoic acid (Formula (III); FIG. 7).

In the third step of the reaction procedure, the disulfide portion of L-prolyl lipoic acid was converted into a dithiol moiety to produce 1-(6,8-dimercaptooctanyl)pyrrolidine-2-carboxylic acid (Formula (IV)) as shown in FIG. 8. To perform this conversion of L-prolyl lipoic acid into a dithiol derivative, L-prolyl lipoic acid, 0.15 g, was dissolved in 25 mL of ethanol and stirred well for 5 minutes. Sodium borohydride (37 mg) was added to the solution by portions over a 2 hour period. Next, in order to derive the crude product, the ethanol was evaporated under reduced pressure, followed by treatment with saturated ammonium chloride (15 mL). The organic compound in the aqueous phase was extracted with dichloromethane (2×50 mL) and evaporated to dryness under reduced pressure. The resultant dimercapto-derivative was purified on column chromatography to get the compound in a purified form.

To confirm the presence of the dithiol compound, the dithiol compound was tested by reacting it with nitric oxide gas as the characteristic feature of thiol compounds includes the formation of a nitroso-derivative upon reaction with nitric oxide gas. To perform this analysis, the intermediate was dissolved in ethanol (25 mL) and cooled to −20° C. using dry ice and acetone. Simultaneously, nitric oxide gas was generated by reacting sodium nitrite and concentrated hydrochloric acid. The nitric oxide gas was then passed through the dithiol solution for two hours. The solution turned a deep pink color which is characteristic of an S-nitroso compound. UV-Visible spectrum analysis of this deep pink solution further showed two characteristic absorption peaks at 330 and 540 nm, confirming the presence of the dithiol compound.

### Example 3

### Synthesis of 1-(6,8-Dimercaptooctanyl)Piperidine-2-Carboxylic Acid

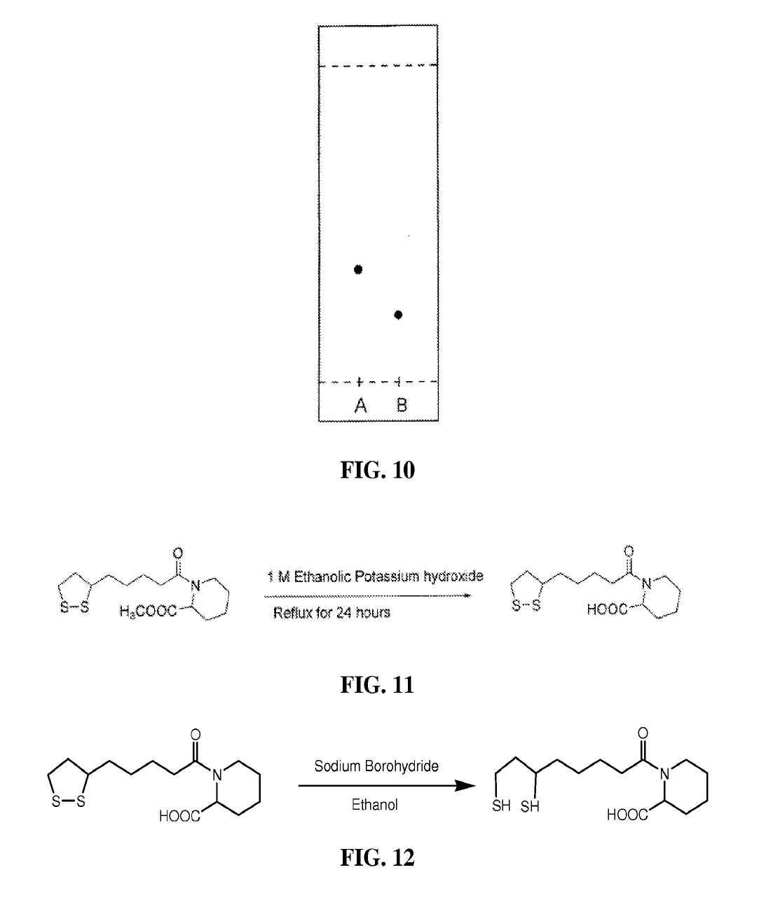

The synthesis of 1-(6,8-Dimercaptooctanyl)Piperidine-2-Carboxylic Acid was performed by a three step procedure that began with the synthesis of (DL)-pipecolinyl methyl ester lipoic acid from (DL)-pipecolinyl methyl ester and (DL)-alpha-lipoic acid. Briefly, the synthesis of (DL)-pipecolinyl methyl ester lipoic acid was completed by combining (DL)-pipecolinic acid methyl ester and (DL)-alpha-lipoic acid using a suitable coupling reagent, as shown in FIG. 9. The first step of the reaction was carried out under a nitrogen atmosphere. (DL)-alpha-lipoic acid, 0.206 g (1 mM); (DL)-pipecolinic acid methyl ester hydrochloride, 0.166 g (1 mM); dimethylaminopyridine, 0.122 g (1 mM); and triethylamine, 0.101 g (1 mM, 0.140 mL) were dissolved in methylene chloride (35 mL) and were combined in a 100 mL round bottom flask. The contents of the flask were stirred well for 5 minutes at room temperature. The coupling reagent EDCI, 0.287 g (1.5 mM) was added in portions over a period of 1.5 hours while the contents of the flask were simultaneously stirred. Completion of the reaction was monitored using thin layer chromatography by comparing the disappearance of the starting materials and the formation of the new product as shown in FIG. 10.

After stirring overnight, the methylene chloride was evaporated under reduced pressure using a rotary evaporator. The resultant crude product was purified using fluorescent preparative thin layer chromatography with a ratio of 90:5:10 ethylacetate:hexane:methanol. The desired compound band was scraped and the compound was extracted from the band by continuously eluting with ethylacetate (350 mL). The solvent was evaporated to dryness under a vacuum. A pale yellow, highly viscous liquid was obtained in a 59% yield, and was determined to be (DL)-pipecolinyl methyl ester lipoic acid (Formula (V)).

In the second step of the reaction procedure, (DL)-Pipecolinyl lipoic acid was synthesized by de-esterification of (DL)-pipecolinyl methyl ester lipoic acid in 1M ethanolic potassium hydroxide under reflux condition as shown in FIG. 11. Briefly, the reaction was carried out under nitrogen atmosphere and (DL)-pipecolinyl methyl ester lipoic acid, 0.158 g (0.5 mM) was placed in a 50 mL round bottom flask equipped with reflux condenser. 25 mL of 1 mM ethanolic potassium hydroxide was added and the mixture was allowed to reflux for 24 hours. Progress of the reaction was monitored by thin layer chromatography. After the overnight reflux, ethanol was removed under reduced pressure followed by the addition of 30 mL of water and extracted the aqueous phase with dichloromethane (2×25 mL). Aqueous phase was carefully transferred to 100 mL conical flask, cooled well on crushed ice and acidified with 1 N hydrochloric acid till pH of the solution is acidic. The aqueous phase was then extracted with dichloromethane (2×50 mL), washed with brine and dried over anhydrous magnesium sulphate and filtered. Solvent was evaporated under reduced pressure to get yellow colored semi-solid 75% yield that was determined to be (DL)-pipecolinyl lipoic acid (Formula (VI)).

In the third step of the reaction procedure, 1-6,8-dimercaptooctanyl)piperidine-2-carboxylic acid (Formula (VII)) was synthesized from pipecolinyl lipoic acid as shown in FIG. 12. To perform this step of the reaction procedure, L-pipecolinyl lipoic acid, 0.165 g, was first dissolved in 25 mL of ethanol and stirred well for 5 minutes. Sodium borohydride (37 mg) was then added to stepwise to the solution over a 2 hour period. Next, in order to derive the crude product, the ethanol was evaporated under reduced pressure, followed by treatment with saturated ammonium chloride (15 mL). The organic compound in the aqueous phase was extracted with dichloromethane (2×50 mL) and evaporated to dryness under reduced pressure. The resultant dimercapto derivative was purified on column chromatography to get the compound in a purified form.

Again, because the characteristic feature of thiol compounds includes the formation of a nitroso-derivative upon reaction with nitric oxide gas, the resulting dithiol compound was tested to confirm its presence by reacting the compound with nitric oxide gas. Again, briefly, the intermediate was dissolved in ethanol (25 mL) and cooled to −20° C. using dry ice and acetone. Simultaneously, nitric oxide gas was generated by reacting sodium nitrite and concentrated hydrochloric acid. The nitric oxide gas was then passed through the dithiol solution for two hours. The solution turned a deep pink color which is characteristic of an S-nitroso compound. UV-Visible spectrum analysis of this deep pink solution showed two characteristic absorption peaks at 330 and 540 nm, confirming the presence of the dithiol compound.

### Example 4

### In Vitro Inhibition of Angiotensin Converting Enzyme Activity

To determine whether the compounds of the presently-disclosed subject matter were capable of reducing the enzymatic activity of Angiotensin Converting Enzyme (ACE), an ACE assay was carried out according to the standard protocols (see, e.g., Schwager, et al. A high-throughput fluorimetric assay for angiotensin I-converting enzyme, Nature Protocols, 2006; 1 (4): 1961-1964, which is incorporated herein by this reference). The ACE assay involved a fluorimetric procedure in which the substrate hippury-L-histidyl-L-leucine (HHL) was used, and the fluorescent adduct of the enzyme-catalyzed product L-histidyl-L-leucine was quantified fluorimetrically. Further, the assay was also adapted for a 96-well plate format to produce comparable data and greater efficiency with respect to both time and reagents. The results from the assay were analyzed in a 96-well plate reader with each concentration measured in triplicate.

Briefly, in a total of 250 μL assay volume, 10 μL of ACE enzyme (Sigma Chemical Co., St. Louis, Mo.) and 10 μL of either 1-(6,8-dimercaptooctanyl)pyrrolidine-2-carboxylic acid or prolyl lipoic acid (Formula (III) was added. Both compounds were incubated with enzyme at 37° C. for 30 min. The ACE substrate HHL, 6 μL in buffer, was then added to each well followed by incubation at 45° C. for 30 min. 170 μL of 0.3M sodium hydroxide solution was added to above solution followed by the addition of phalaldehyde solution in methanol. The mixture was then allowed to shake for 5 min. This step was followed by the addition of 32 μL of 2N hydrochloric acid to each well and subsequent measurement of the fluorescence at an excitation wavelength of 360 nm and an emission wavelength of 480 nm. Three controls were used to compare the readings, with the controls including wells without the enzyme, wells without the compounds, and wells without the HHL enzymatic substrate.

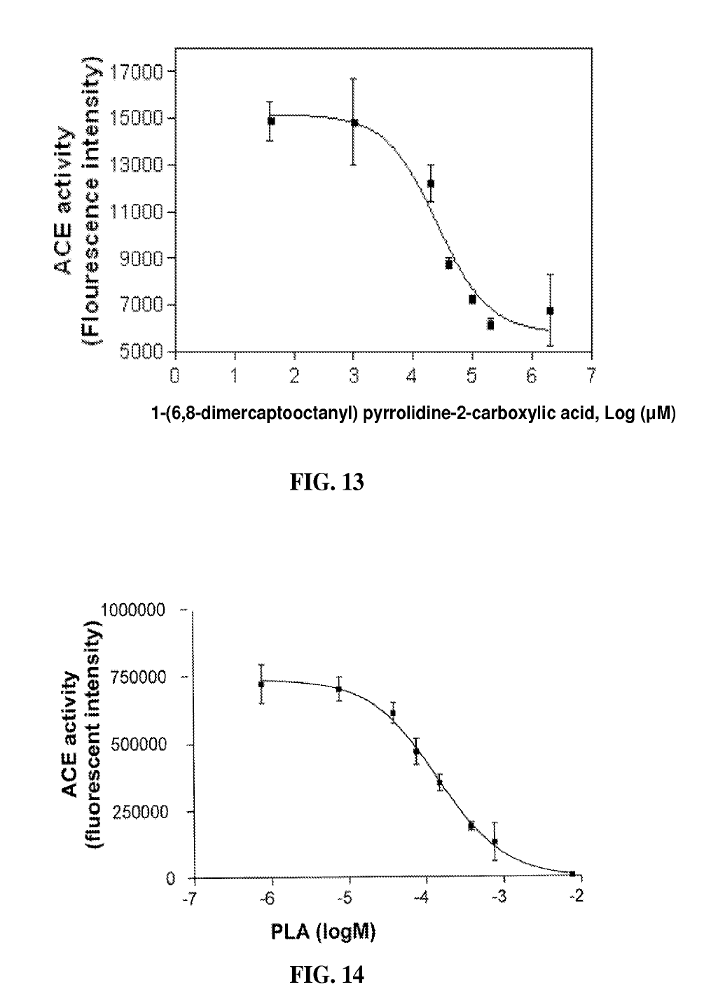

Upon analysis of the results, it was observed that both 1-(6,8-dimercaptooctanyl)pyrrolidine-2-carboxylic acid and prolyl lipoic acid (Formula (III) effectively reduced the formation of the enzyme-catalyzed product, L-histidyl-L-leucine, and were thus shown to effectively reduce ACE activity, indicating that the compounds of the present invention are useful in a method of reducing ACE activity. Furthermore, the IC50 value of the compounds were calculated by plotting fluorescence counts against the various concentrations of the compounds used in the experiments, as shown in FIG. 13 (1-(6,8-dimercaptooctanyl)pyrrolidine-2-carboxylic acid) and in FIG. 14 (prolyl lipoic acid (Formula (III)).

Based on these graphs, the IC50 value of 1-(6,8-dimercaptooctanyl)pyrrolidine-2-carboxylic acid was initially found to be approximately 29 μM. To further examine the IC50 value in relation to the absolute amounts of thiol groups of the 1-(6,8-dimercaptooctanyl)pyrrolidine-2-carboxylic acid compound, however, the amount of thiol groups in the reaction mixtures was also measured according to Ellman's method (see, e.g. Simpson R J, Estimation of free thiols and disulfide bonds using Ellman's reagent, CSHL Press, Cold Spring Harbor, N.Y., 2003; and, Ellman G L, Tissue sulfhydral groups, Arch. Biochem. Biophys., 1959; 82: 70-77, each of which are incorporated herein by this reference). Briefly, the method utilized 5,5′-dithiobis-(2-nitrobenzoic acid) or DTNB as a chemical for measuring the amount of thiol groups, as the thiols react with that compound, cleaving the disulfide bond to give 2-nitro-5-thiobenzoate (TNB⁻), which then ionizes to the TNB²⁻ dianion in water at neutral and alkaline pH. The resulting TNB²⁻ ion has a yellow color, and the reaction is rapid and stoichiometric, with the addition of one mole of thiol releasing one mole of TNB. The TNB²⁻ can then be quantified in a spectrophotometer by measuring the absorbance of visible light at 412 nm, using an extinction coefficient of 14,150M⁻¹ cm⁻¹ for dilute buffer solutions, and a coefficient of 13,700 M⁻¹ cm⁻¹ for high salt concentrations, such as 6 M guanidinium hydrochloride or 8 M urea. In this regard, the reaction protocol thus involved 300 μL of total assay volume containing multiple concentrations of 1-(6,8-dimercaptooctanyl)pyrrolidine-2-carboxylic acid in buffer followed by the addition of the DTNB reagent in buffer (sodium phosphate, pH 8.0). The reaction was done in triplicate measurement. The absorbance was measured at 412 nm. Based on the thiol quantification by Ellman's method, the 1-(6,8-dimercaptooctanyl)pyrrolidine-2-carboxylic acid compound included oxidized compounds (e.g., disulphide, metal particles and insoluble salts, etc.), and, after thiol quantification, the 1050 value for 1-(6,8-dimercaptooctanyl)pyrrolidine-2-carboxylic acid was recalculated to be 6.6 μM.

### Example 5

### Effect of Lipoic Acid and Prolyl Lipoic Acid on Vasodilation

To determine the effect of lipoic acid and prolyl lipoic acid (Formula (III)) on vasodilation, aortas from male Sprague-Dawley Rats (n=2) were utilized. Briefly, each animal was first sacrificed and their aortas were removed. The aortas were then incubated in phosphate-buffered-saline (PBS) containing 44 mM glucose for 4 hours. Eight 5-mm ring segments of thoracic aorta were then suspended in a myograph chamber filled with a Krebs buffer of the following composition: NaCl, 118.3; KCl, 4.69; CaCl₂, 1.87; MgSO₄, 1.20; K₂HPO₄, 1.03; NaHCO₃ 25.0; and glucose 11.1; all values in mM, pH 7.40. During the following hour, the resting tension was increased to optimize constrictions due to exposure to KCl. The vessel segments were then pre-constricted with phenylephrine (PE; 0.1 μM) and were subjected to prolyl lipoic acid (Formula (III)) or lipoic acid. Vasodilation in the rat aortic rings in response to L-prolyl lipoic acid (1×10⁻⁴ M) or lipoic acid (1×10⁻⁴ M) (both dissolved in aqueous media) was then measured after a 4-hour incubation with glucose (44 mM). Endothelial cell function in the aortic rings was also measured by pre-constricting the aortic rings to the EC₅₀ of phenylephrine and then subjecting the rings to a maximum acetylcholine dose (10⁻⁵M).

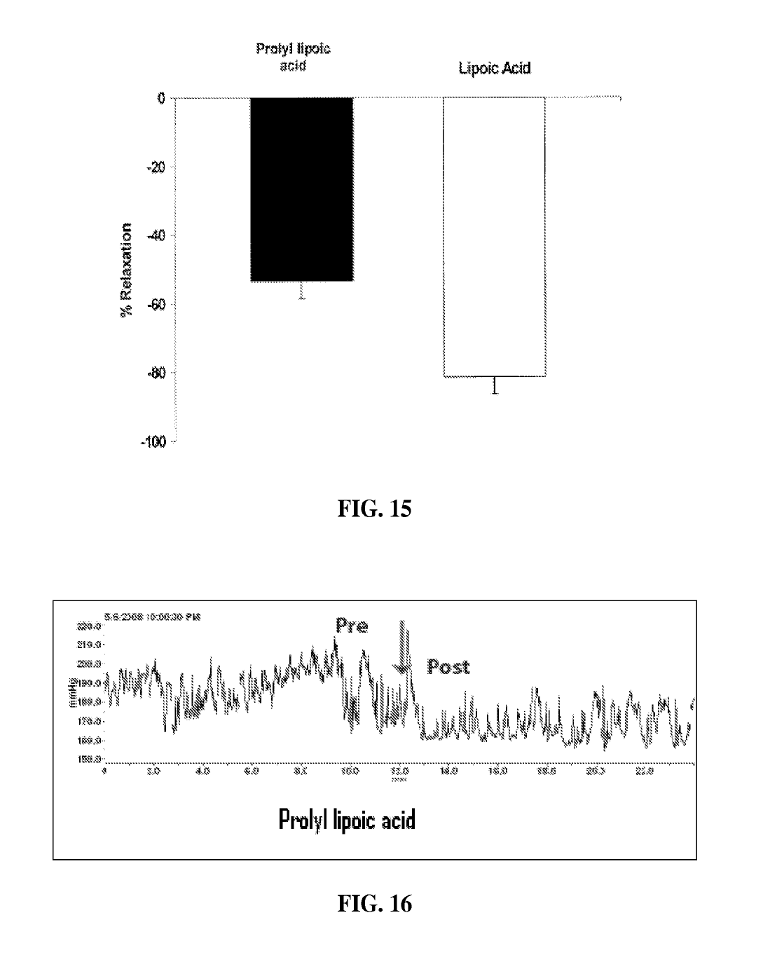

Upon analysis of the results, it was observed that both lipoic acid and prolyl lipoic acid (Formula (III)) relaxed pre-constricted rat aortic segments at the doses used (FIG. 15), with prolyl lipoic acid causing an increase in relaxation as compared to lipoic acid. Furthermore, prolyl lipoic acid was also shown to improve endothelial function in the aortic rings as evidenced by increased vasodilation following the administration of acetylcholine.

### Example 6

### Effect of Prolyl Lipoic Acid on Hypertension

To determine the effect of prolyl lipoic acid (Formula (III)) in an animal model of hypertension, spontaneously hypertensive stroke prone rats (SHRSP) were first procured from Charles River at the age of 6 weeks (Charles River Laboratories, Wilmington, Mass., U.S.A.). After a period of acclimatization for 4 weeks, the rats were then implanted with intra-arterial catheters in the abdominal aorta along with a radio-telemetry monitoring device to assess systolic blood pressure. Briefly, to implant the radio-telemetry devices, the operative procedure involved anesthetizing the animals with 375 mg/kg of 2,2,2-tribromoethanol (Sigma Chemical Co., St. Louis, Mo.). An incision was then made in the abdomen and the aorta was exposed. A 0.4 mm catheter attached to a combination pressure transducer, transmitter, and battery, all encapsulated in a implantable micro-miniature electronic monitor (PA-C20, Data Sciences International, St. Paul, Minn.), was then inserted. This capsule-shaped device had a 1.9 cc volume and was previously shown to be effective for chronic blood pressure measurement in mice 25-27 grams and larger.

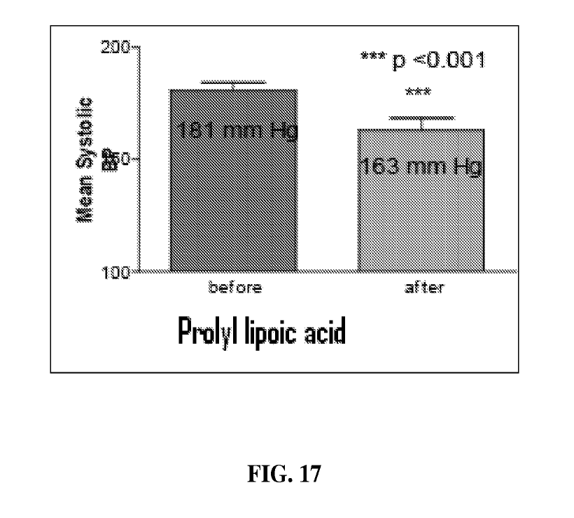

Following implantation of the radio-telemetry devices, the animals were allowed to recover and the aortic pressure of each animal was monitored continuously. Once the animals sufficiently recovered, the rats then received 50 mg/kg of prolyl lipoic acid, dissolved in aqueous media, in a single dose. The blood pressures were then measured over a period of 12 hours and compared to the previous 12 hour period. Upon analysis of the results, it was observed that prolyl lipoic acid significantly reduced blood pressure in an acute manner after one dose in the genetic model of hypertension (FIGS. 16-17).

To further examine the effects of prolyl lipoic acid (Formula (III)) on hypertension, the effect of prolyl lipoic acid was examined in spontaneously hypertensive rats over a longer time period. Briefly, spontaneously hypertensive rats (SHRSP rats, 10 weeks of age, n=10/group) were randomized to placebo or prolyl lipoic acid (50 mg/kg) groups for a duration of 6 weeks. The animals were housed 2 to a cage and were given Harlan-Teklad Laboratory Rodent Diet ad libitum (Harlan Laboratories, Indianapolis, Ind.). Prolyl lipoic acid or saline placebo was administered to the rats by daily intraperitoneal (IP) dosing.

During the 6 week experimental period, blood pressure measurements were performed in a sub-group of 4 animals randomized to prolyl lipoic acid by intra-arterial catheter placement in the infra-renal aorta and acute administration of the prolyl lipoic acid prior to administration of drug. Additionally, four SHRSP rats (12 weeks old) were implanted with radio-telemetry transmitters (DataSciences Inc., St. Paul, Minn.) into their abdominal aortas and blood pressure was measured using the DSI system (DataSciences Inc.). In these later experiments, blood pressure was recorded continuously over 24-hour periods and reported as the change in blood pressure in the 12-hour period following the administration of the agent.

Following the completion of the six week period, the animals were euthanized at week 17 by lethal pentobarbital injection and tissues were removed from the animal. Upon removal of the tissues, endothelial function was assessed. It is known that endothelial function can refer to the unimpaired production of or availability of NO and/or a balance in the relative contribution of endothelium-derived relaxing and contracting factors (such as ET-1, angiotensin, and oxidants). Indeed, endothelial dysfunction in diabetes may result from a decreased bioavailability of NO (secondary to insulin resistance) coupled with an exaggerated production of ET-1 (stimulated by hyperinsulinemia or hyperglycemia). Endothelial dysfunction has also been implicated in the pathogenesis and clinical course of all known cardiovascular diseases and is associated with future risk of adverse cardiovascular events. As such, once the tissues were removed from the animals, endothelial cell function was assessed in the experimental groups by myography as percent-relaxation to acetylcholine or sodium nitroprusside subsequent to pre-contraction with 0.3 μM phenylephrine.

Furthermore, inflammatory gene expression was assessed in the aorta, heart, brain and kidney by probing for mRNA transcript levels of the various inflammatory genes. Aortic and cardiac collagen synthesis was also assessed by Picrosirius Red staining and Massom-Trichrome staining and quantified by an automated thresholding program.

Additionally, four to eight sections of each organ were stained with CD68 or H&E for 10 animals (5 in each group) and images were obtained at 200× by bright-field microscopy. MetaMorph software was used for automated thresholding. The percentage of thresholded area of the picture was recorded and reported. Additionally, about 12 organ sections per rat were prepared for 10 of the rats (5 in each group). These sections were 5 microns in size and were stained with hematoxylin and eosin (H&E). Images were obtained at 40× (using bright-field microscopy and analyzed using MetaMorph software. For each image, the external elastic lamina of the aorta was traced using a drawing tool, and this area was logged. The luminal area was also measured. Thus, the tunica intima and tunica media were included in these measurements, not tunica adventitia. The wall thickness was calculated by subtracting the luminal area from the wall area.

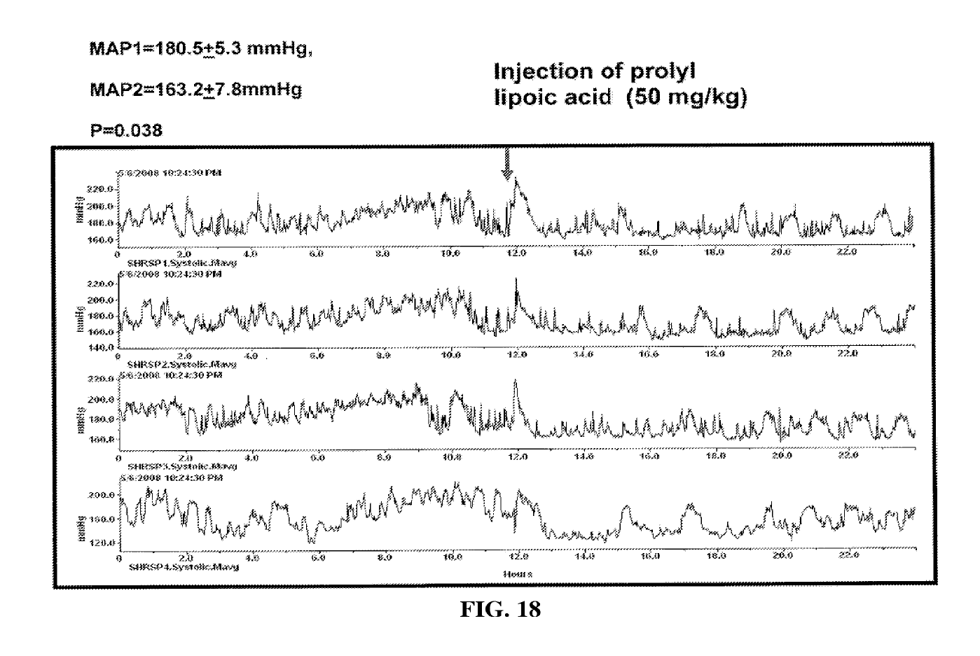

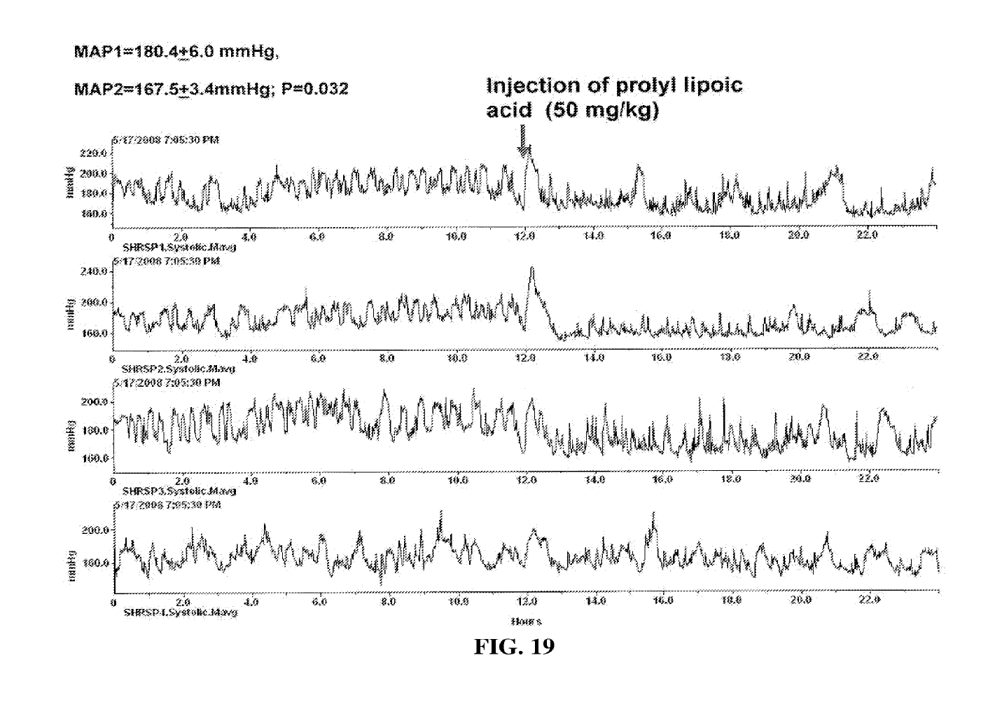

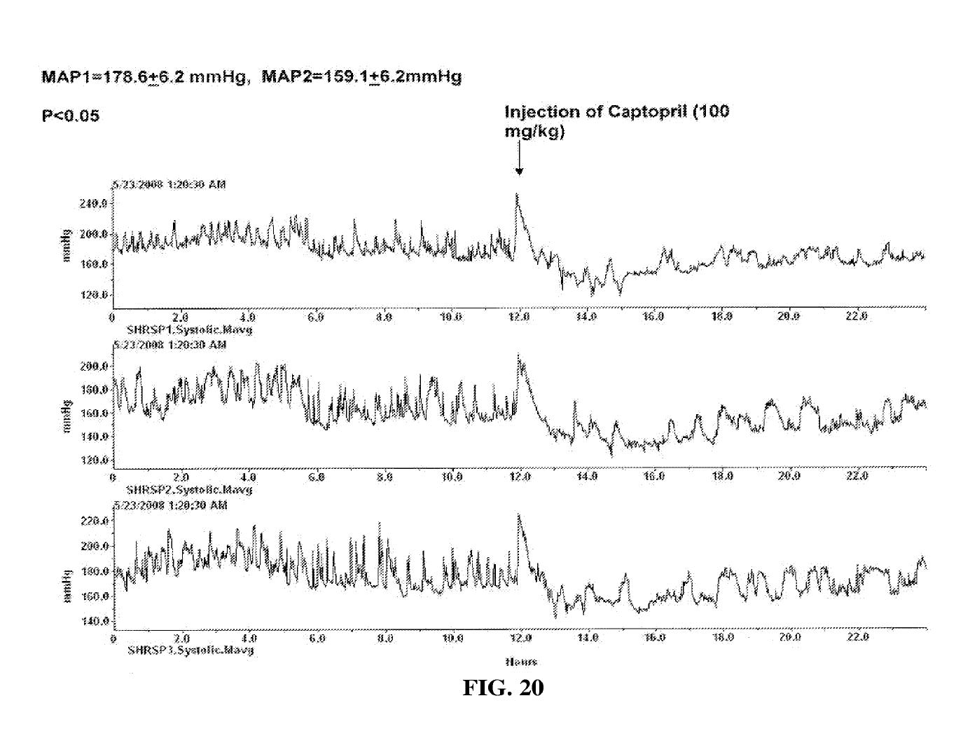

Upon analysis of the results from the foregoing experiments, it was observed that prolyl lipoic acid (Formula (III)) acutely lowered blood pressure at a dose of 50 mg/kg. These effects of prolyl lipoic acid on blood pressure (FIGS. 18-19) were comparable to the angiotensin converting enzyme inhibitor, captopril (FIG. 20).

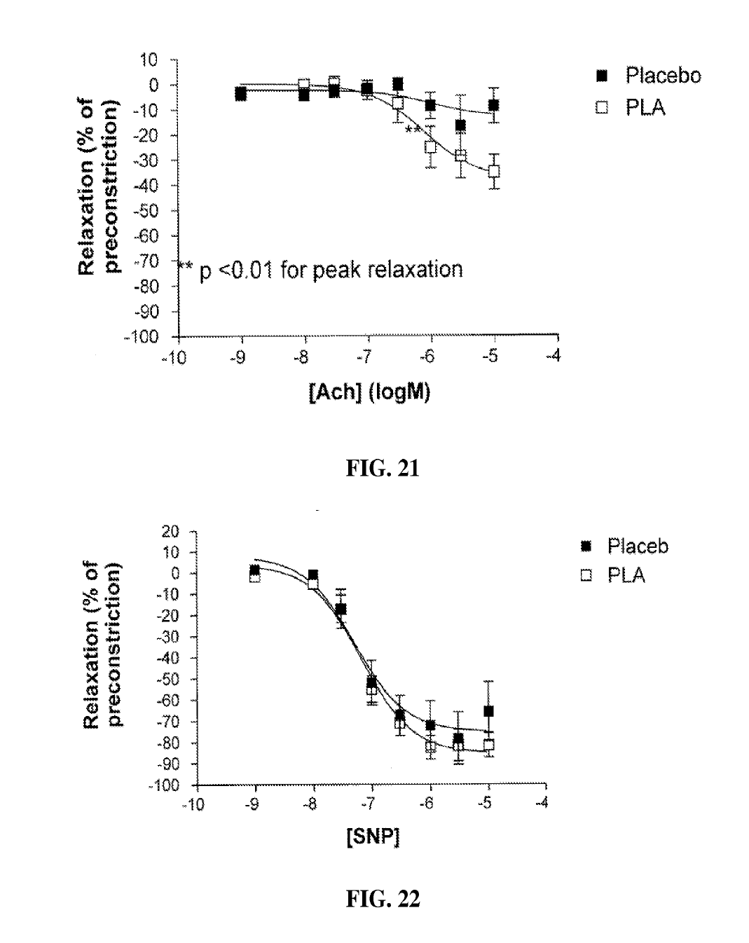

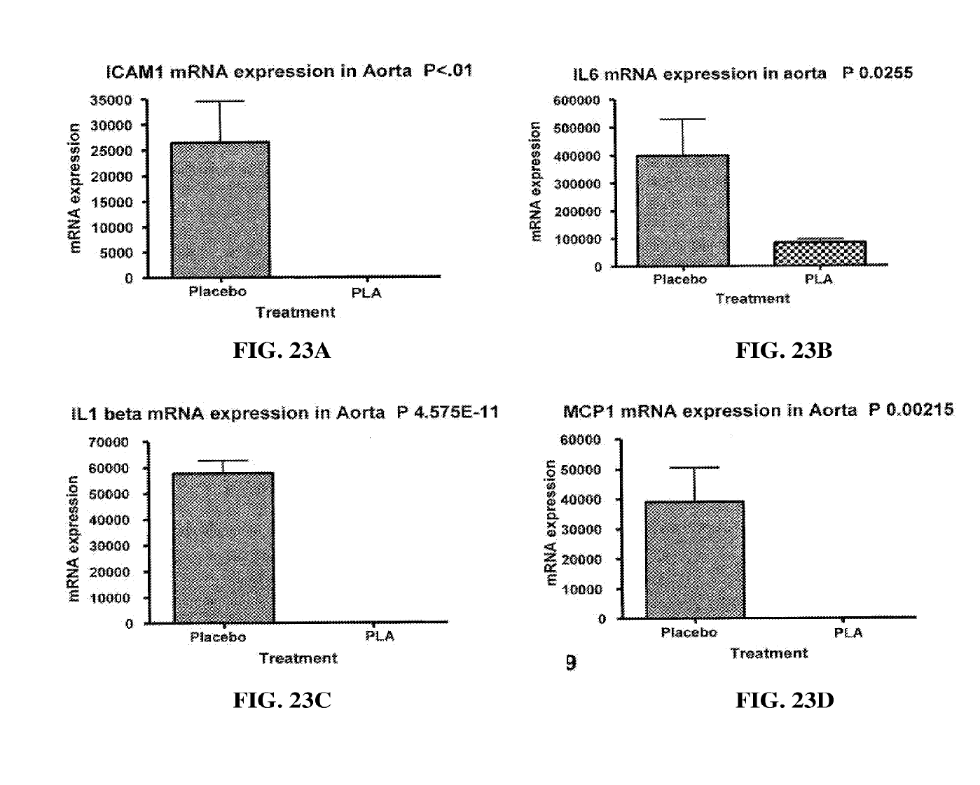

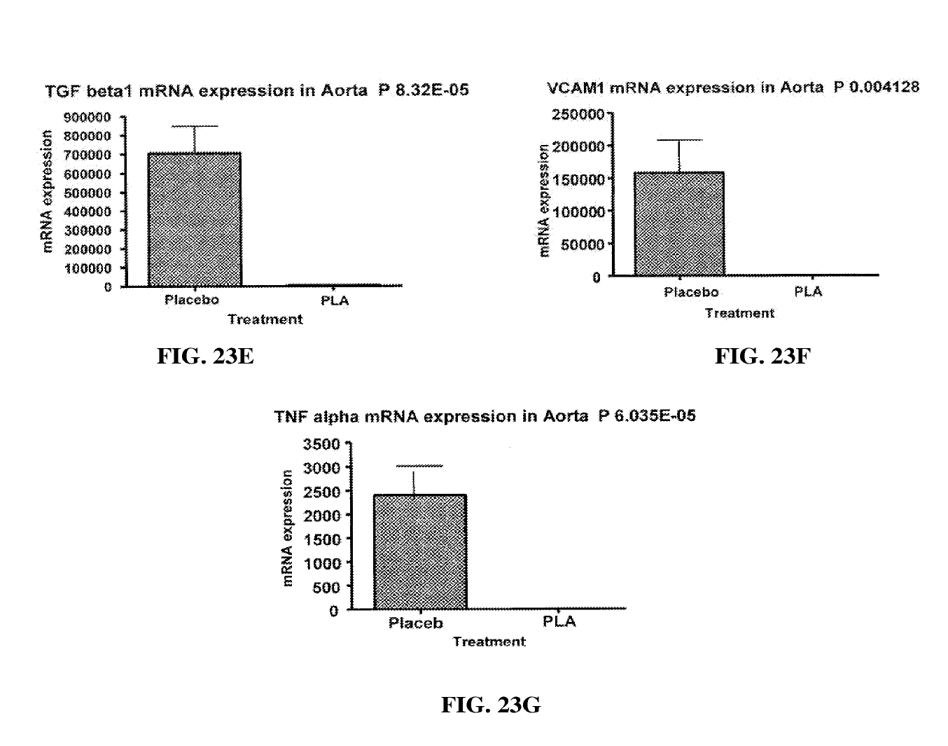

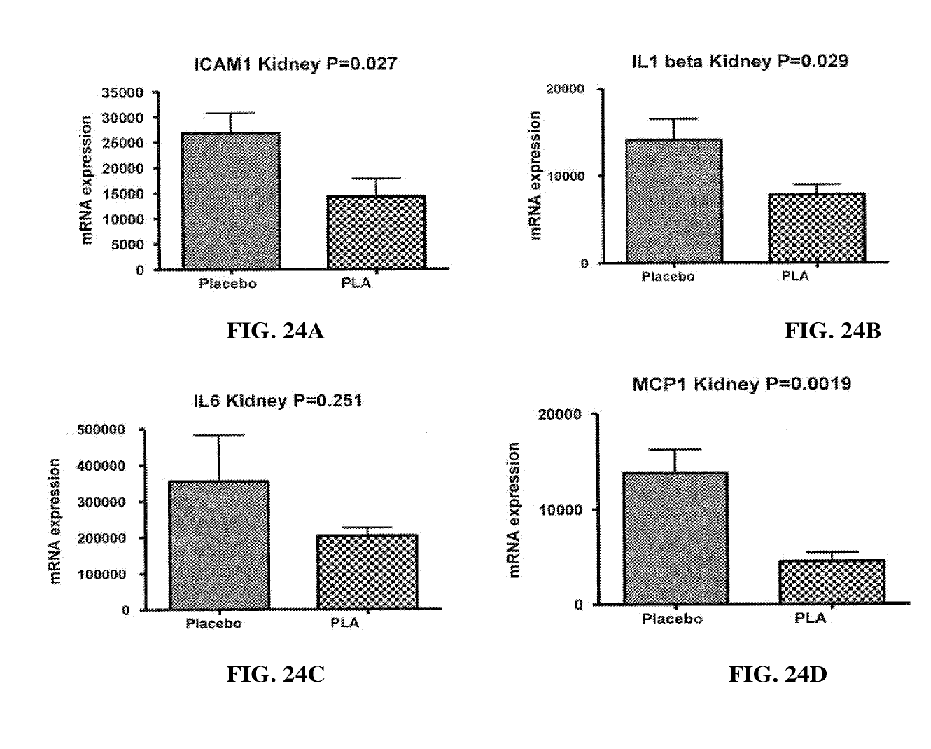

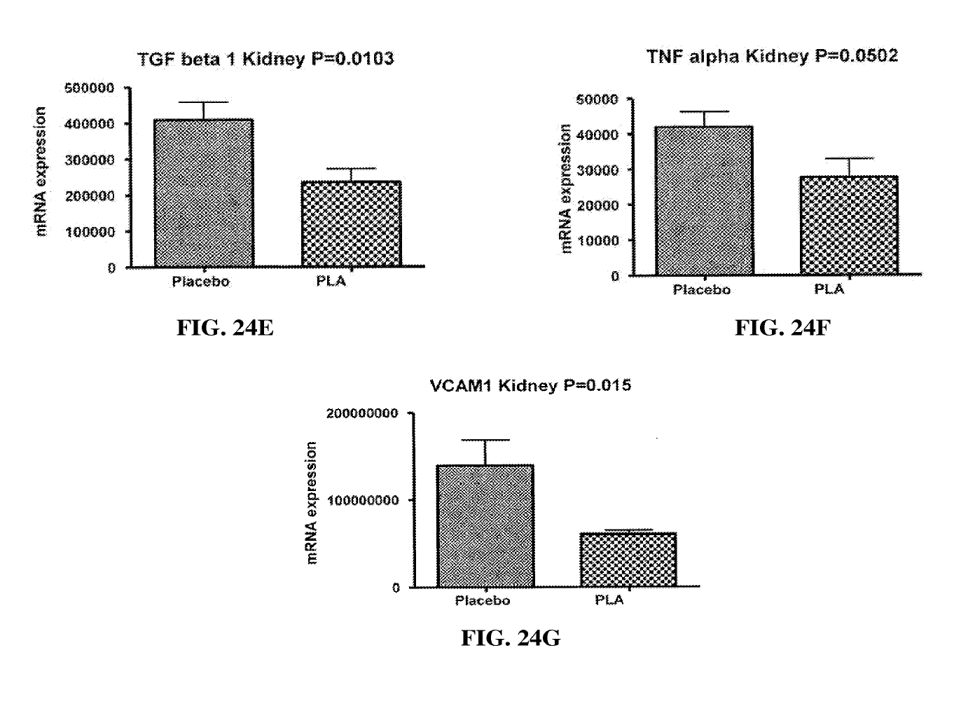

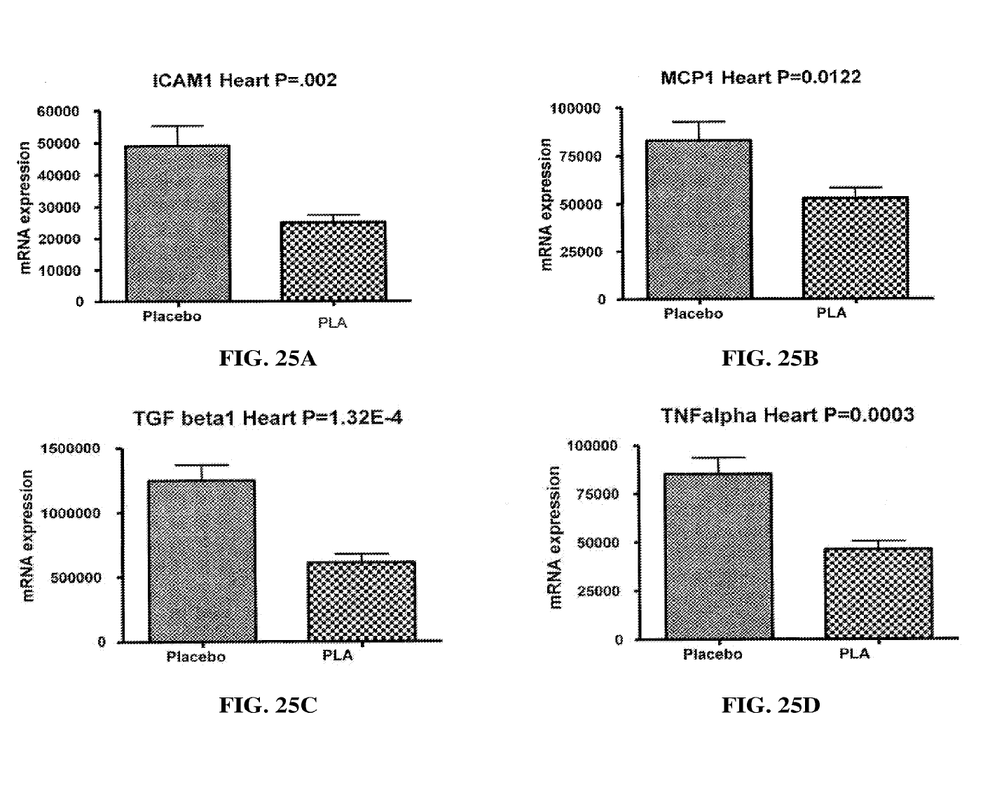

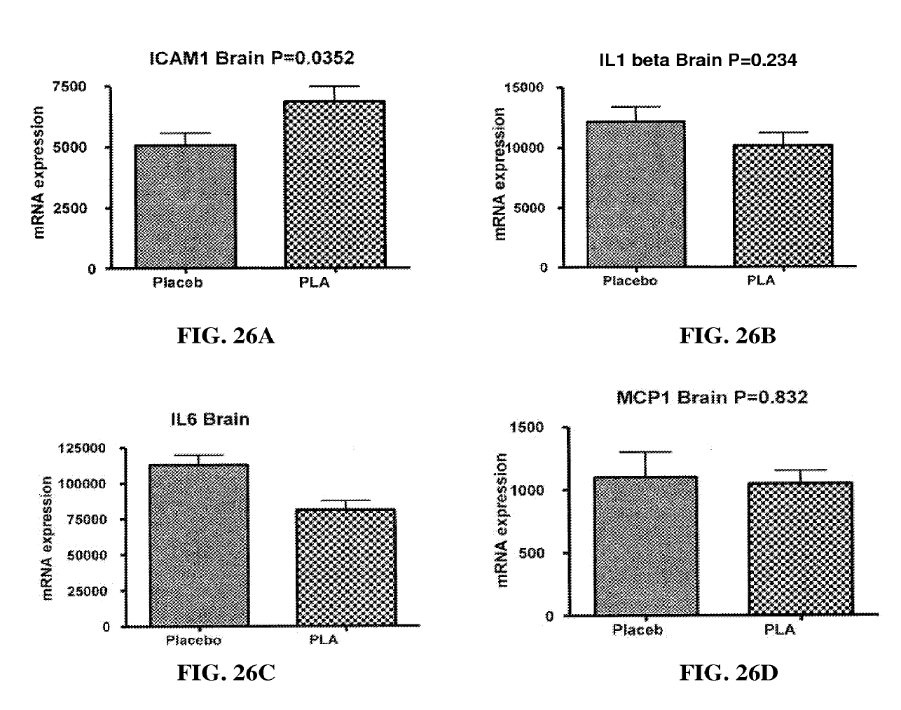

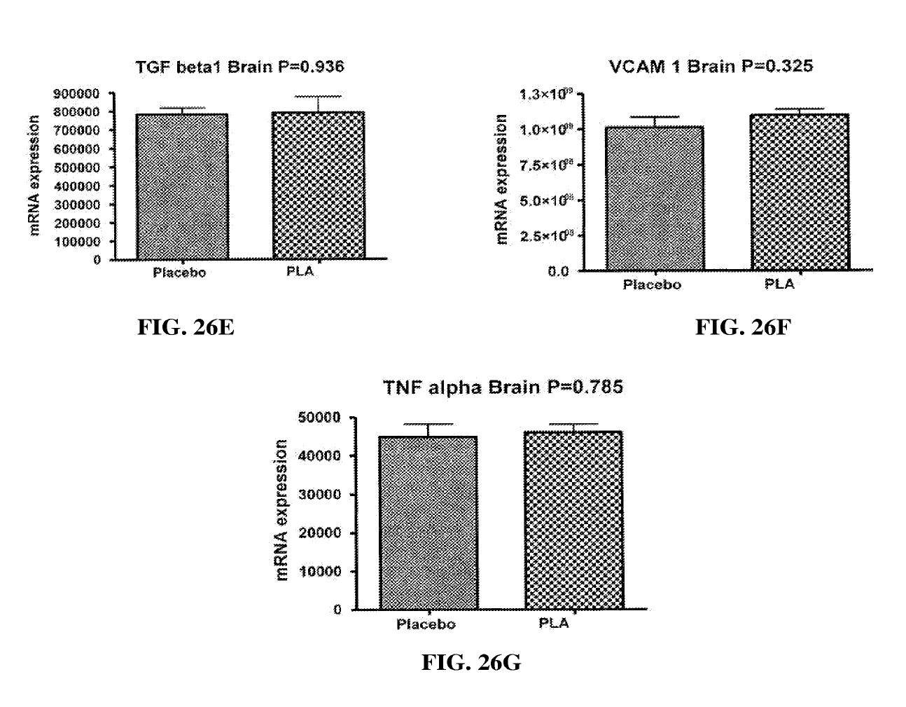

Upon analysis of the tissue samples from the experimental groups, it was also observed that prolyl lipoic acid (Formula (III)) at a dose of 50 mg/kg improved endothelial function after 6 weeks of treatment in the SHRSP model (FIGS. 21-22). Prolyl lipoic acid also markedly reduced inflammatory gene expression in aorta (FIG. 23A-G), kidney (FIG. 24A-G) and heart (FIG. 25A-D) tissues. The marked reduction in inflammatory gene expression observed in the foregoing tissues was not as pronounced in the brain tissue samples (FIGS. 26A-G); however, some reductions in expression in brain tissues were observed for ICAM1 and IL-6 (FIGS. 26A and 26C, respectively).

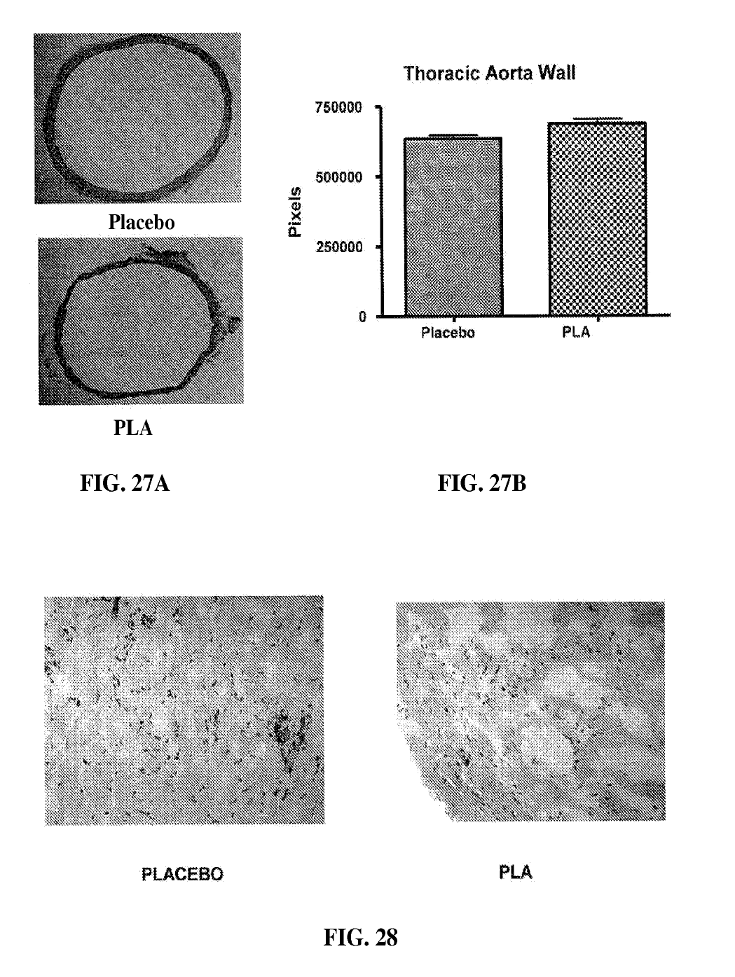

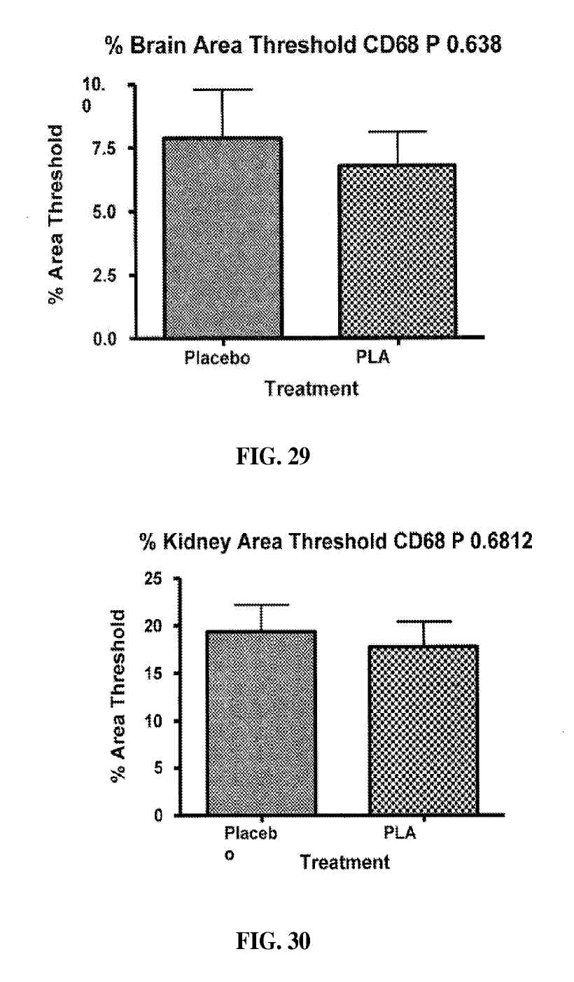

Prolyl lipoic acid (Formula (III)) also had favorable effects on inflammatory cell recruitment to the heart and kidney. Furthermore, the results demonstrated that prolyl lipoic acid had effects in reducing aortic medial hypertrophy (FIGS. 27A-B) without affecting CD68 expression in the myocardium, brain or kidneys (FIGS. 28-30), which is indicative macrophage recruitment to those tissues and organs.

It will be understood that various details of the present invention can be changed without departing from the scope of the subject matter disclosed herein. Furthermore, the foregoing description is for the purpose of illustration only, and not for the purpose of limitation.

What is claimed is:

1. A compound having the Formula (I): 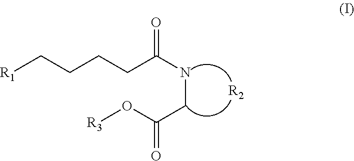
wherein:
R₁ is selected from the group consisting of 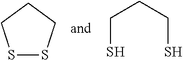
R₂ completes a 5- or 6-membered non-aromatic heterocyclic ring structure; and
R₃ is selected from the group consisting of CH₃ and H;
or a pharmaceutically acceptable salt or solvate thereof.

2. The compound of claim 1, wherein the compound has the Formula (II):

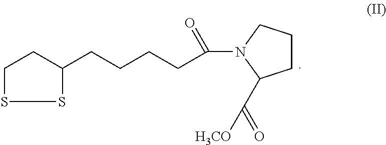

3. The compound of claim 1, wherein the compound has the Formula (III):

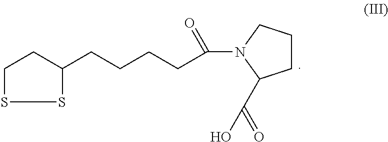

4. The compound of claim 1, wherein the compound has the Formula (IV):

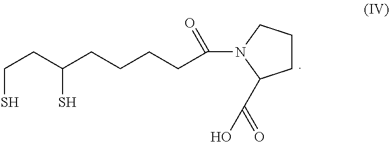

5. The compound of claim 1, wherein the compound has the Formula (V):

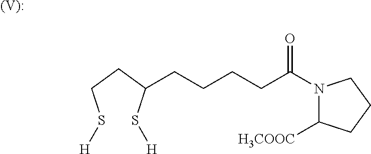

6. The compound of claim 1, wherein the compound has the Formula (VI):

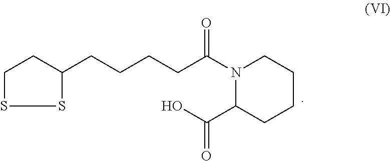

7. The compound of claim 1, wherein the compound has the Formula (VII):

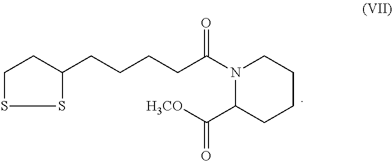

8. The compound of claim 1, wherein the compound has the Formula (VIII):

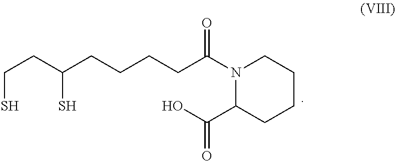

9. The compound of claim 1, wherein the compound has the Formula (IX):

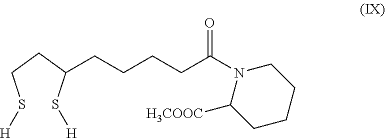

10. A pharmaceutical composition comprising a compound according to claim 1 and a pharmaceutical acceptable vehicle, carrier or excipient.

11. A compound having the Formula (X): 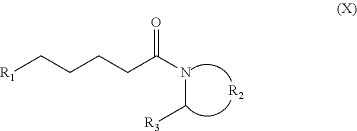
wherein:
R₁ is selected from the group consisting of: 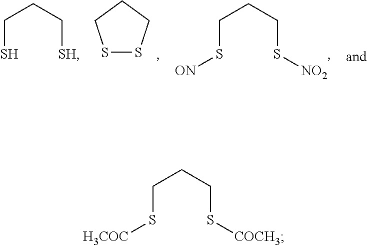
R₂ completes a five- or six-membered non-aromatic heterocyclic ring structure optionally containing a hydroxyl substituent, or an eight- or ten-membered bicyclic aromatic or non-aromatic heterocyclic ring structure; and
R₃ is selected from the group consisting of: 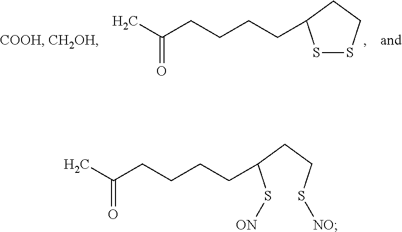
or a pharmaceutically acceptable salt or solvate thereof.

12. The compound of claim 11, wherein the compound has a formula selected from the group consisting of:

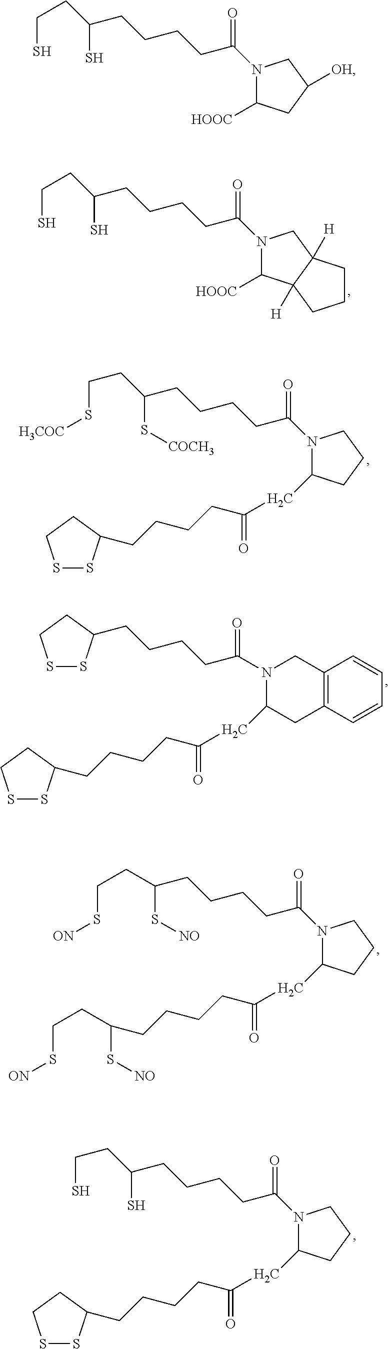

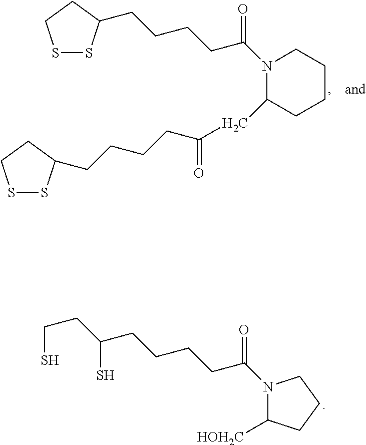

13. A compound having the Formula (XI): 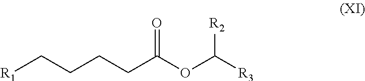
wherein:
R₁ is selected from the group consisting of: 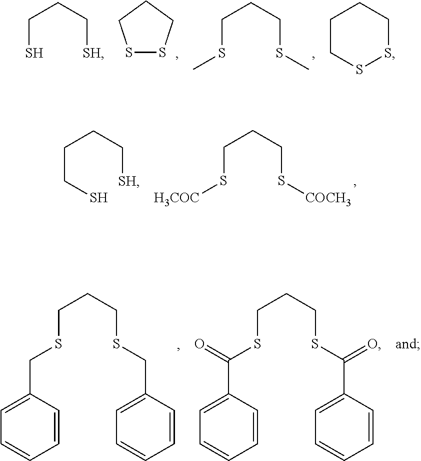
R₂ is selected from the group consisting of:
(CH₂)₂CH₃, (CH₂)₃NH₂, (CH₂)₃OH, (CH₂)₄COOH, 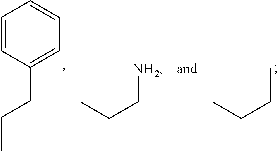
and
R₃ is selected from the group consisting of: 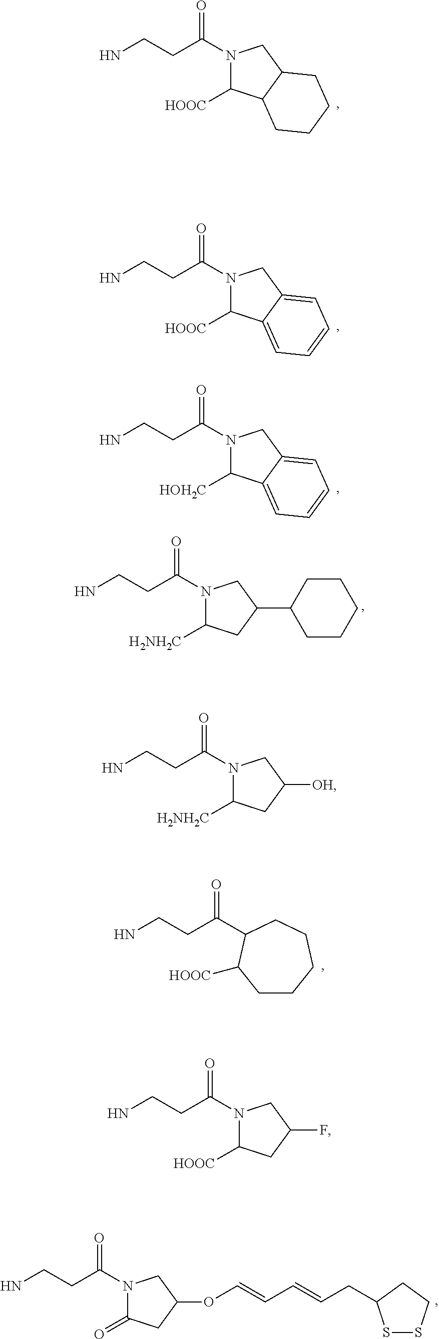 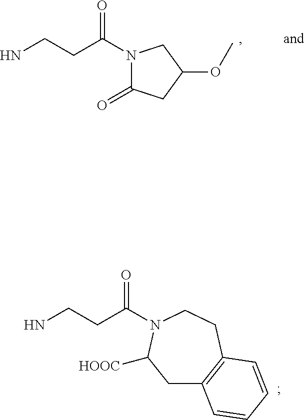
or a pharmaceutically acceptable salt or solvate thereof.

14. The compound of claim 13, wherein the compound has a formula selected from the group consisting of:

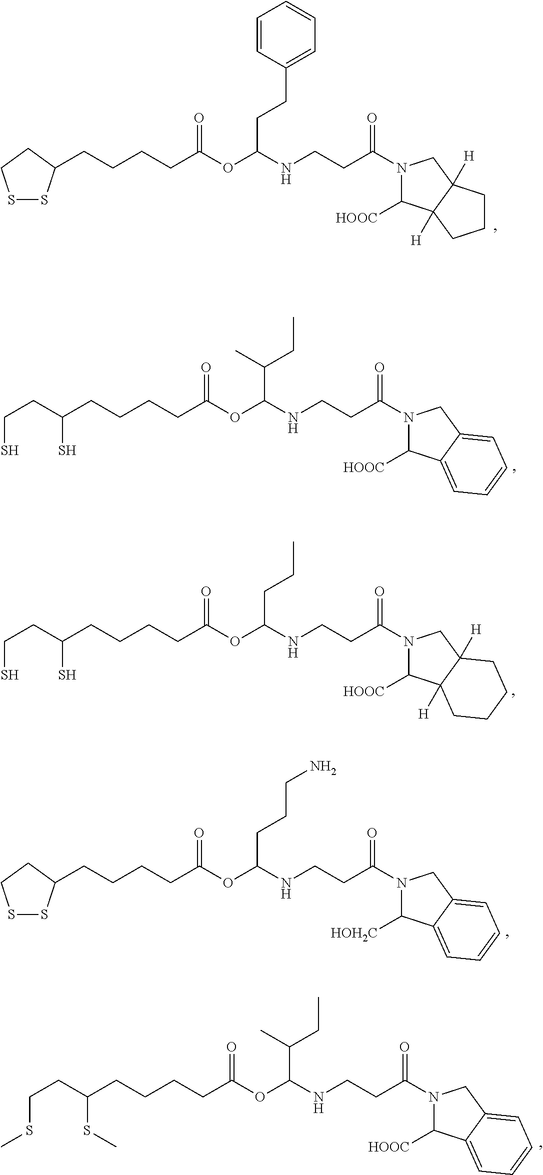

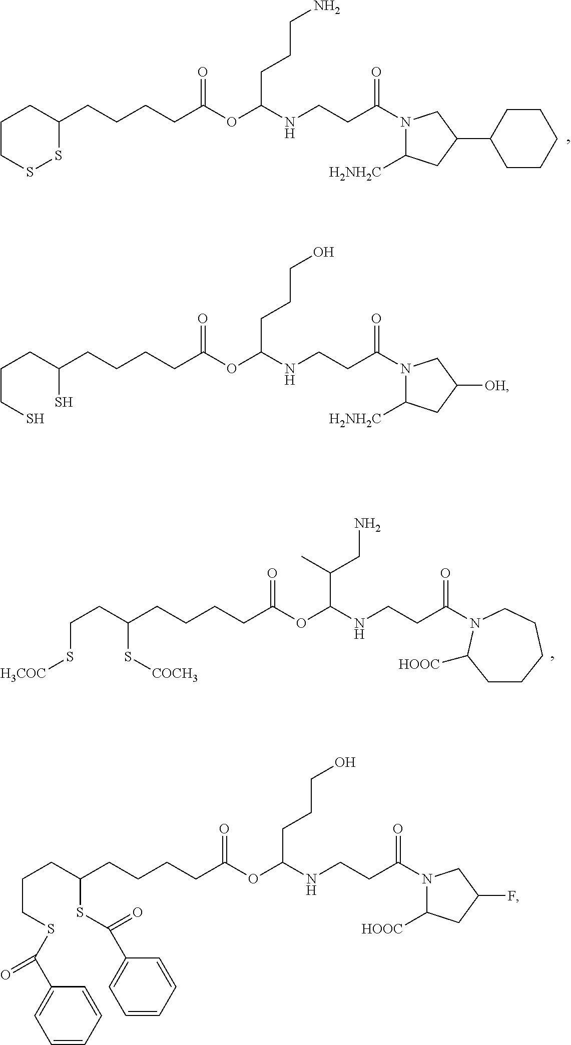

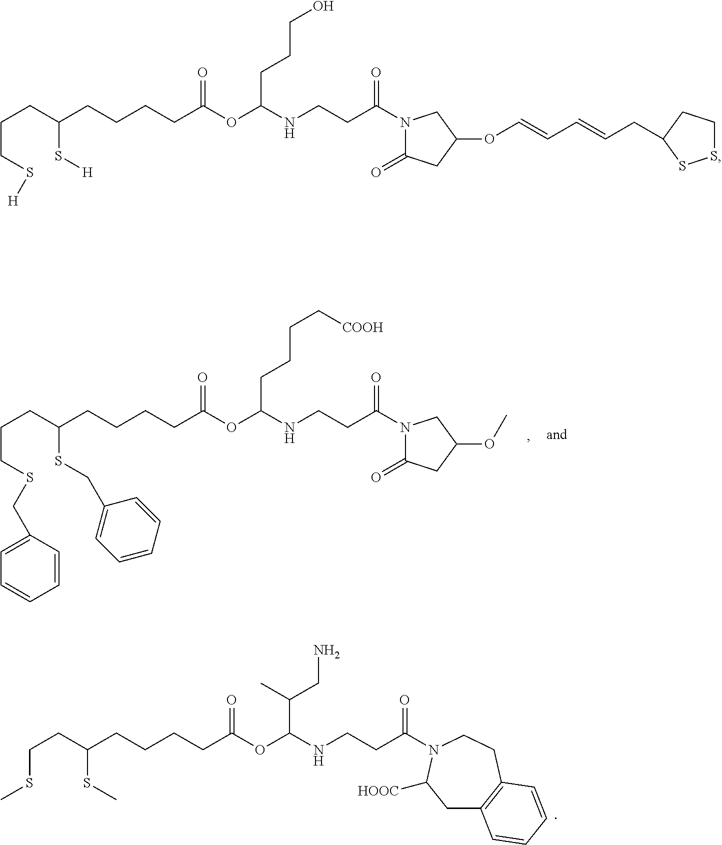

15. A compound having the Formula (XII): 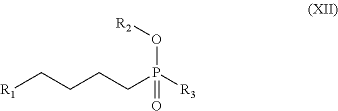
wherein:
R₁ is selected from the group consisting of: 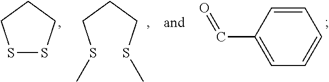
R₂ is selected from the group consisting of: 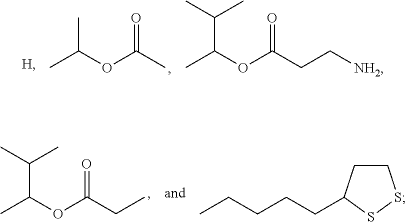
and
R₃ is selected from the group consisting of: 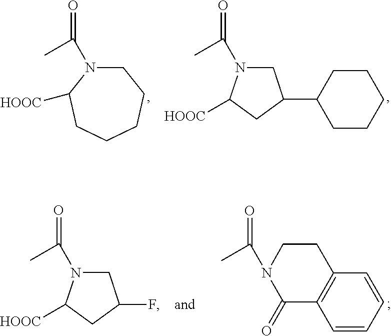
or a pharmaceutically acceptable salt of solvate thereof.

16. The compound of claim 15, wherein the compound has a formula selected from the group consisting of:

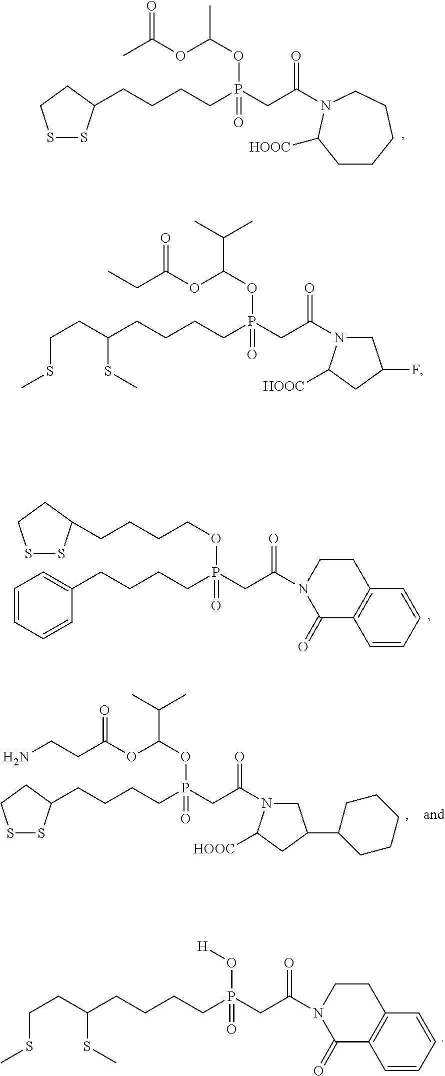

17. A compound having the Formula XIII: 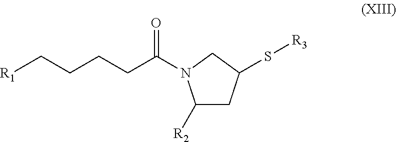
wherein:
R₁ is selected from the group consisting of: 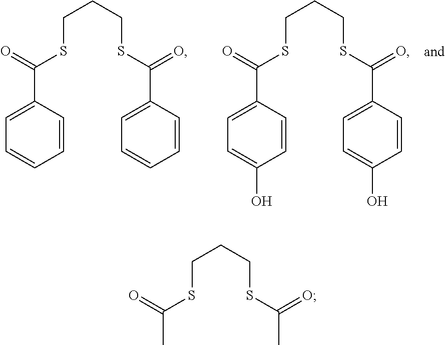
R₂ is selected from the group consisting of:
COOH, 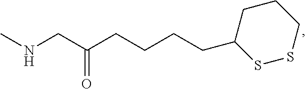
and CH₂OCH₃; and
R₃ is selected from the group consisting of: H, CH₃, and COCH₃;
or a pharmaceutically acceptable salt or solvate thereof.

18. The compound of claim 17, wherein the compound has a formula selected from the group consisting of:

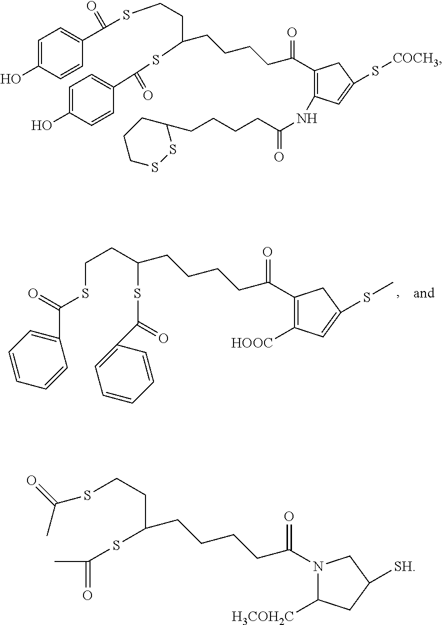

19. A compound having the Formula XIV: 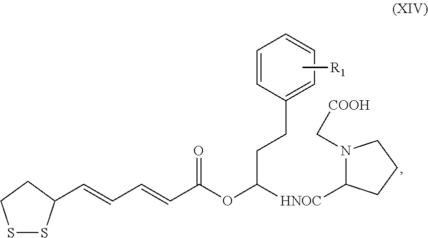
wherein R₁ is selected from the group consisting of OH, NO₂, NH₂, and OCH₃; or
a pharmaceutically acceptable salt or solvate thereof.

20. A compound having the Formula XV: 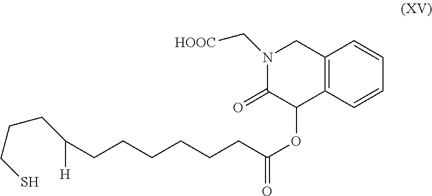
or a pharmaceutically acceptable salt or solvate thereof.

21. A compound having the Formula XVI: 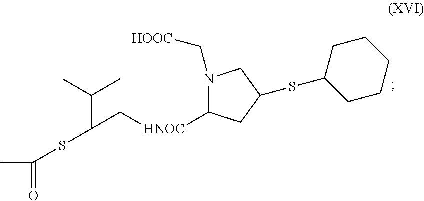
or a pharmaceutically acceptable salt or solvate thereof.

22. A compound having the Formula XVII: 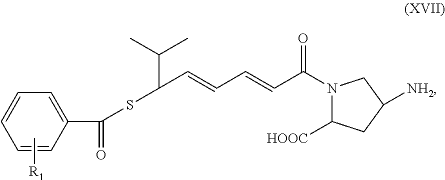
wherein R₁ is selected from the group consisting of OH, NO₂, OCH₃, and NH₂; or
a pharmaceutically acceptable salt or solvate thereof.

23. A compound having the Formula (XVIII): 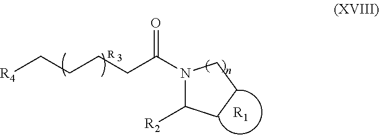
wherein R₁ is an optional ring that can be selected from the group consisting of a cyclopentyl ring, a cyclohexyl ring, a phenyl ring, and a substituted phenyl ring where the substituents on the phenyl ring are selected from the group consisting of OH, OCH₃, NH₂, NO₂, COOH;
wherein R₂ is selected from the group consisting of a free carboxyl, methyl or ethyl ester, a primary alcohol, and a primary amine group;
wherein n is an integer from 1 to 3, and the nitrogen-containing ring is a substituted or unsubstituted ring selected from the group consisting of a pyrrolidine ring, a piperidine ring, and a saturated azepine ring;
wherein R₃ is an alkyl group that includes from 1 to 3 carbon atoms, and which can include one or more conjugated or unconjugated double bonds; and
wherein R₄ is a substituted or unsubstituted 5- or 6-membered disulphide moiety or a 5- or 6-membered dithiol moiety selected from the group consisting of

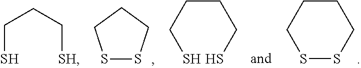

24. A method of reducing angiotensin converting enzyme activity, comprising contacting an angiotensin converting enzyme with an effective amount of a compound selected from the group consisting of the following Formulas (I) and (X)-(XVIII), or pharmaceutically acceptable salts or solvates thereof: 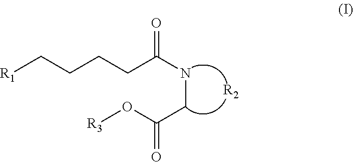
wherein:
R₁ is selected from the group consisting of 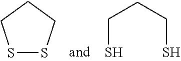
R₂ completes a 5- or 6-membered non-aromatic heterocyclic ring structure; and
R₃ is selected from the group consisting of CH₃ and H; 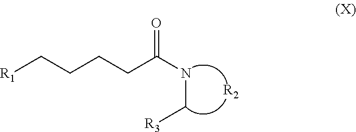
wherein:
R₁ is selected from the group consisting of: 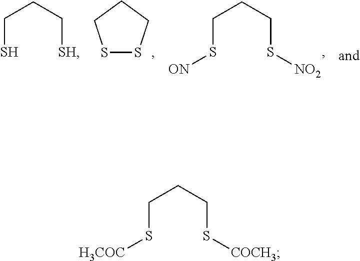
R₂ completes a five- or six-membered non-aromatic heterocyclic ring structure optionally containing a hydroxyl substituent, or an eight- or ten-membered bicyclic aromatic or non-aromatic heterocyclic ring structure; and
R₃ is selected from the group consisting of: 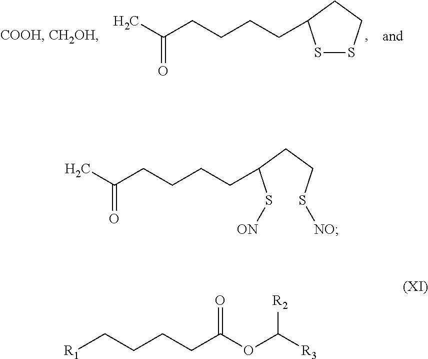
wherein:
R₁ is selected from the group consisting of: 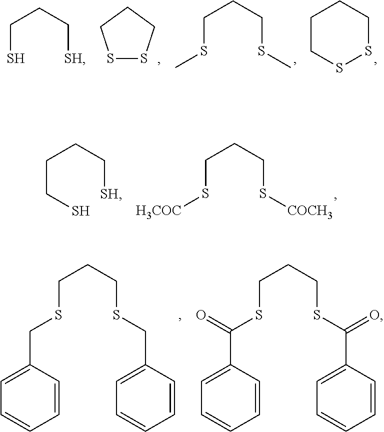
and;
R₂ is selected from the group consisting of:
(CH₂)₂CH₃, (CH₂)₃NH₂, (CH₂)₃OH, (CH₂)₄COOH, 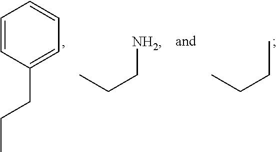
and
R₃ is selected from the group consisting of: 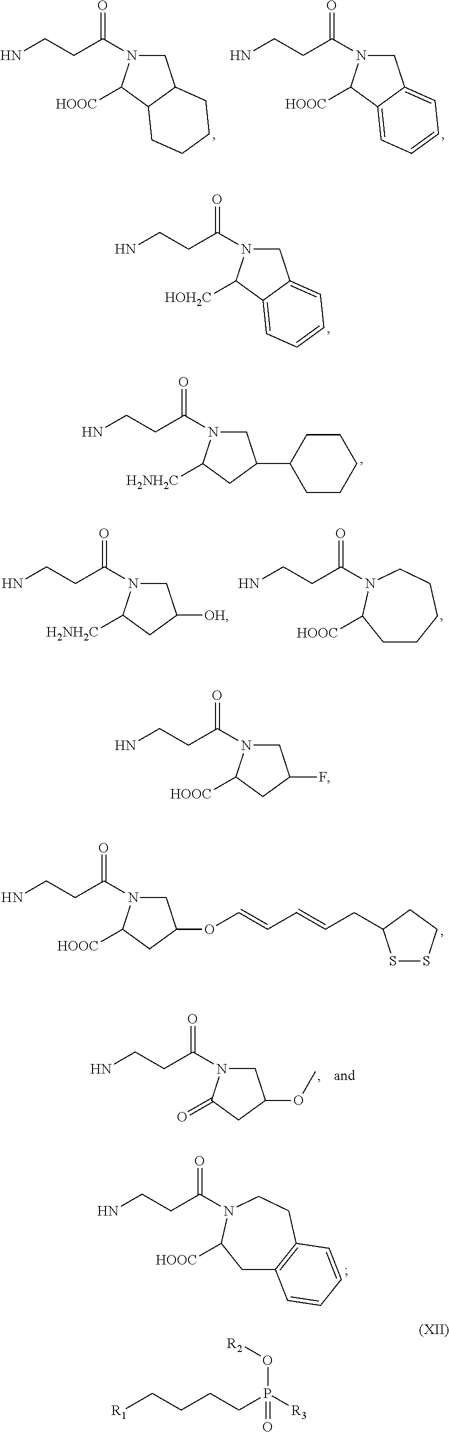
wherein:
R₁ is selected from the group consisting of: 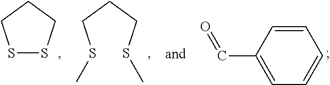
R₂ is selected from the group consisting of: 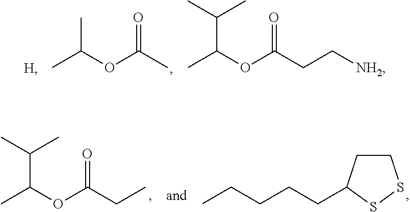
and
R₃ is selected from the group consisting of: 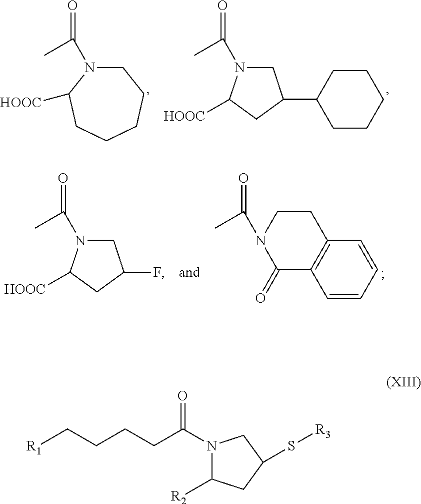
wherein:
R₁ is selected from the group consisting of: 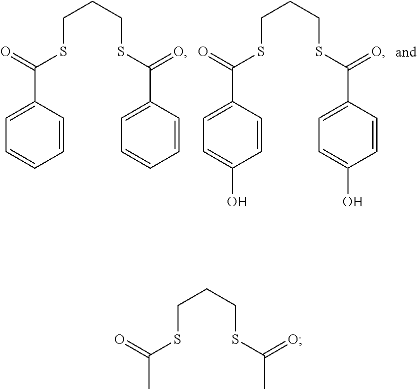
R₂ is selected from the group consisting of:
COOH, 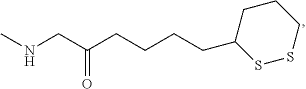
and CH₂OCH₃; and
R₃ is selected from the group consisting of: H, CH₃, and COCH₃; 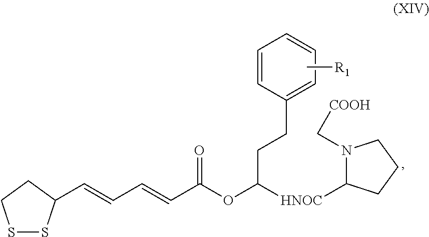
wherein R₁ is selected from the group consisting of OH, NO₂, NH₂, and OCH₃; 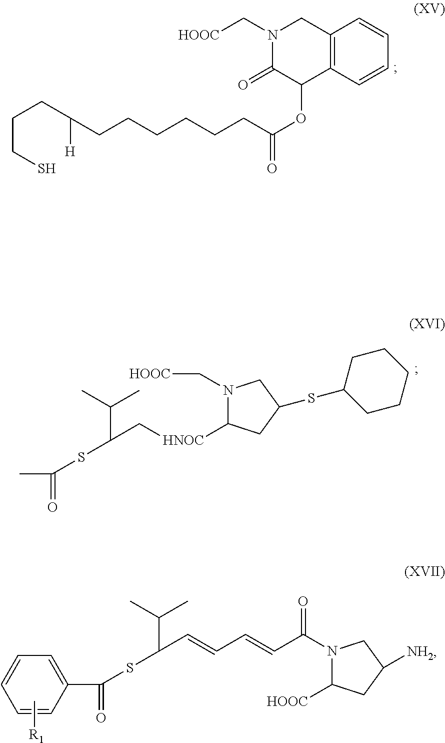
wherein R₁ is selected from the group consisting of OH, NO₂, OCH₃, and NH₂; and 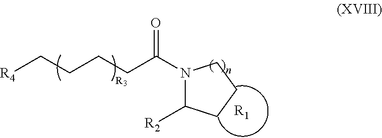
wherein R₁ is an optional ring that can be selected from the group consisting of a cyclopentyl ring, a cyclohexyl ring, a phenyl ring, and a substituted phenyl ring where the substituents on the phenyl ring are selected from the group consisting of OH, OCH₃, NH₂, NO₂, COOH;
wherein R₂ is selected from the group consisting of a free carboxyl, methyl or ethyl ester, a primary alcohol, and a primary amine group;
wherein n is an integer from 1 to 3, and the nitrogen-containing ring is a substituted or unsubstituted ring selected from the group consisting of a pyrrolidine ring, a piperidine ring, and a saturated azepine ring;
wherein R₃ is an alkyl group that includes from 1 to 3 carbon atoms, and which can include one or more conjugated or unconjugated double bonds; and
wherein R₄ is a substituted or unsubstituted 5- or 6-membered disulphide moiety or a 5- or 6-membered dithiol moiety selected from the group consisting of

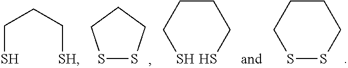

25. The method of claim 24, wherein the angiotensin converting enzyme is contacted with a concentration of the compound in the range of about 1 to about 200 nM.

26. The method of claim 24, wherein the angiotensin converting enzyme is contacted with an effective amount of the compound by administering to a subject a dose of the compound of about 2.5 to about 400 mg/day.

27. A method of treating a renin-angiotensin aldosterone system-related disorder, comprising administering to a subject in need thereof an effective amount of a compound selected from the group consisting of the following Formulas (I) and (X)-(XVIII), or pharmaceutically acceptable salts or solvates thereof: 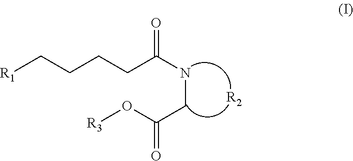
wherein:
R₁ is selected from the group consisting of 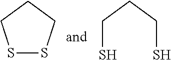
R₂ completes a 5- or 6-membered non-aromatic heterocyclic ring structure; and
R₃ is selected from the group consisting of CH₃ and H; 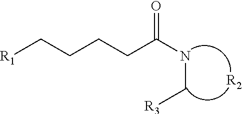
wherein:
R₁ is selected from the group consisting of: 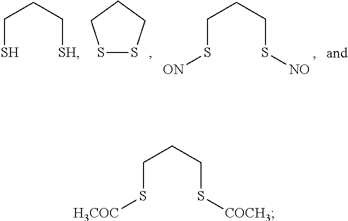
R₂ completes a five- or six-membered non-aromatic heterocyclic ring structure optionally containing a hydroxyl substituent, or an eight- or ten-membered bicyclic aromatic or non-aromatic heterocyclic ring structure; and
R₃ is selected from the group consisting of:
COOH, CH₂OH, 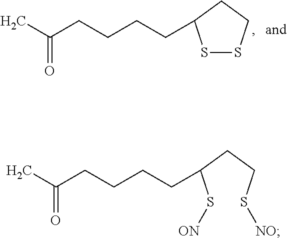 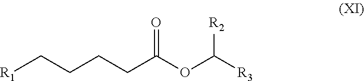
wherein:
R₁ is selected from the group consisting of: 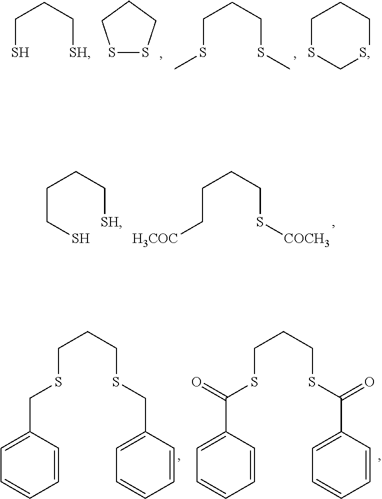
and;
R₂ is selected from the group consisting of:
(CH₂)₂CH₃, (CH₂)₃NH₂, (CH₂)₃OH, (CH₂)₄COOH, 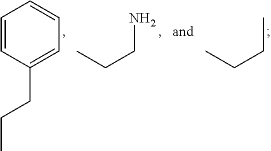
and
R₃ is selected from the group consisting of: 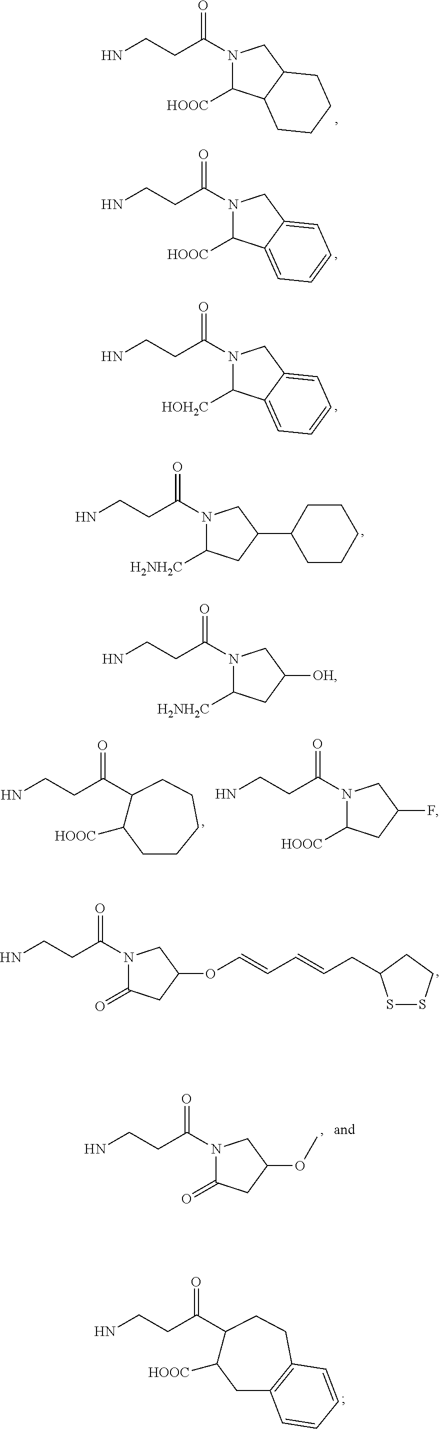 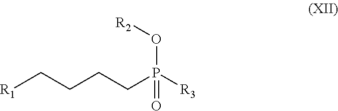
wherein:
R₁ is selected from the group consisting of: 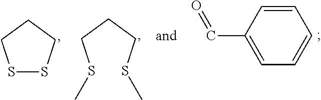
R₂ is selected from the group consisting of: 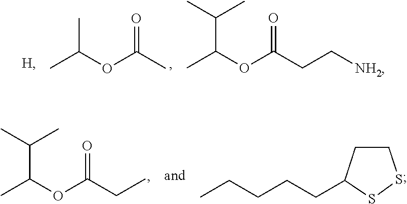
and
R₃ is selected from the group consisting of: 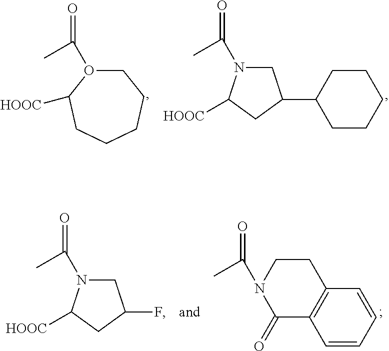 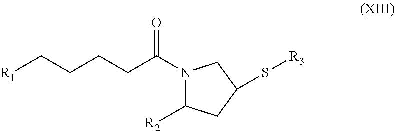
wherein:
R₁ is selected from the group consisting of: 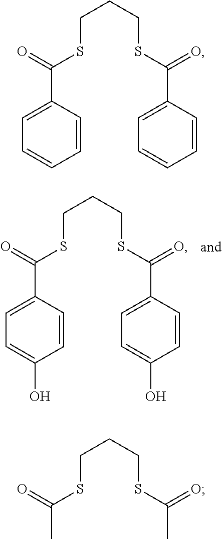
R₂ is selected from the group consisting of:
COOH, 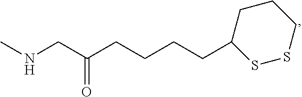
and CH₂OCH₃; and
R₃ is selected from the group consisting of: H, CH₃, and COCH₃; 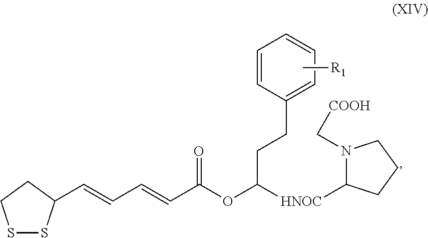
wherein R₁ is selected from the group consisting of OH, NO₂, NH₂, and OCH₃; 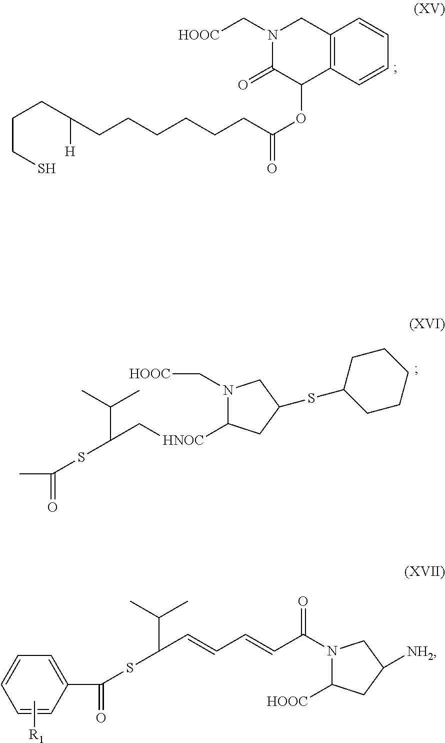
wherein R₁ is selected from the group consisting of OH, NO₂, OCH₃, and NH₂; and 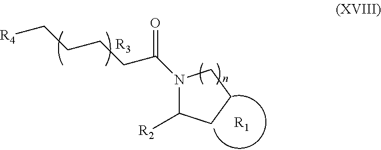
wherein R₁ is an optional ring that can be selected from the group consisting of a cyclopentyl ring, a cyclohexyl ring, a phenyl ring, and a substituted phenyl ring where the substituents on the phenyl ring are selected from the group consisting of OH, OCH₃, NH₂, NO₂, COOH;
wherein R₂ is selected from the group consisting of a free carboxyl, methyl or ethyl ester, a primary alcohol, and a primary amine group;
wherein n is an integer from 1 to 3, and the nitrogen-containing ring is a substituted or unsubstituted ring selected from the group consisting of a pyrrolidine ring, a piperidine ring, and a saturated azepine ring;
wherein R₃ is an alkyl group that includes from 1 to 3 carbon atoms, and which can include one or more conjugated or unconjugated double bonds; and
wherein R₄ is a substituted or unsubstituted 5- or 6-membered disulphide moiety or a 5- or 6-membered dithiol moiety selected from the group consisting of

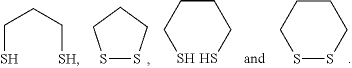

28. The method of claim 27, wherein the renin-angiotensin aldosterone system-related disorder is selected from the group consisting of: hypertension, diabetes mellitus, target organ damage related to diabetes mellitus, atherosclerosis, coronary heart disease, angina, stroke, renal disorders, and Reynaud's disease.

29. The method of claim 27, wherein administering the compound to the subject lowers blood pressure in the subject.

30. The method of claim 27, wherein administering the compound to the subject reduces activity of an angiotensin converting enzyme in the subject.

31. The method of claim 27, wherein administering the compound to the subject reduces an amount of expression of an inflammatory gene in the subject.

32. The method of claim 31, wherein the inflammatory gene is selected from the group consisting of: ICAM1, IL-6, IL-1β, MCP1, TGF-β1, VCAM1, TNF-α, or combinations thereof.

33. The method of claim 31, wherein the amount of expression of an inflammatory gene is reduced in aortic, heart, or kidney tissues of a subject.

34. The method of claim 31, wherein the amount of expression of an inflammatory gene is reduced in brain tissues of a subject, and wherein the inflammatory gene is selected from the group consisting of IL-6 and ICAM1.

35. The method of claim 27, wherein the compound is administered to the subject by a route selected from the group consisting of parenteral, intramuscular, intraperitoneal, subcutaneous, and oral administration.

36. The method of claim 27, wherein the subject is a mammal.

37. The method of claim 36, wherein the mammal is a human.

38. The method of claim 27, wherein the compound is administered to a subject at a concentration of from about 2.5 to about 400 mg per day.

39. A method of reducing hypertension comprising contacting an angiotensin converting enzyme with an effective amount of a compound selected from the group consisting of the following Formulas (I) and (X)-(XVIII), or pharmaceutically acceptable salts or solvates thereof: 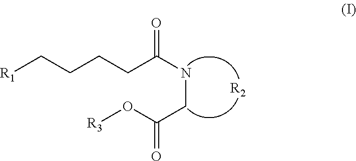
wherein:
R₁ is selected from the group consisting of 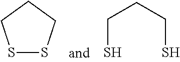
R₂ completes a 5- or 6-membered non-aromatic heterocyclic ring structure; and
R₃ is selected from the group consisting of CH₃ and H; 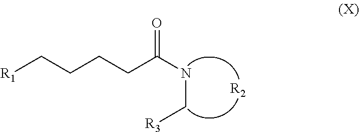
wherein:
R₁ is selected from the group consisting of: 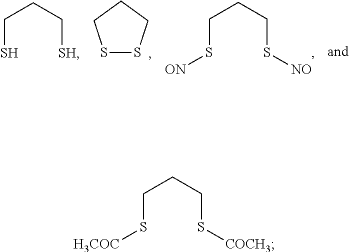
R₂ completes a five- or six-membered non-aromatic heterocyclic ring structure optionally containing a hydroxyl substituent, or an eight- or ten-membered bicyclic aromatic or non-aromatic heterocyclic ring structure; and
R₃ is selected from the group consisting of:
COOH, CH₂OH, 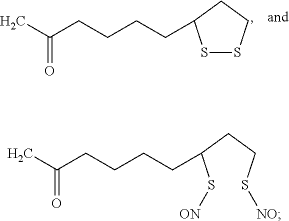 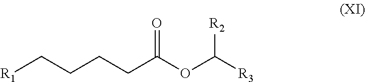
wherein:
R₁ is selected from the group consisting of: 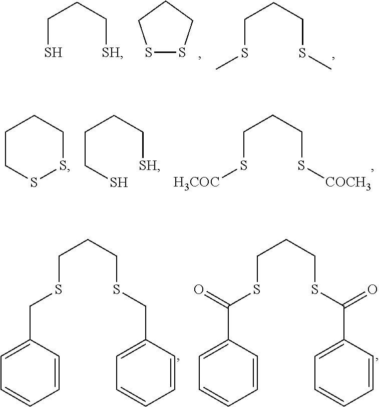
and;
R₂ is selected from the group consisting of:
(CH₂)₂CH₃, (CH₂)₃NH₂, (CH₂)₃OH, (CH₂)₄COOH, 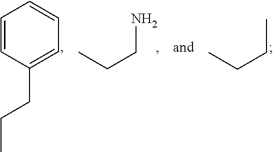
and
R₃ is selected from the group consisting of: 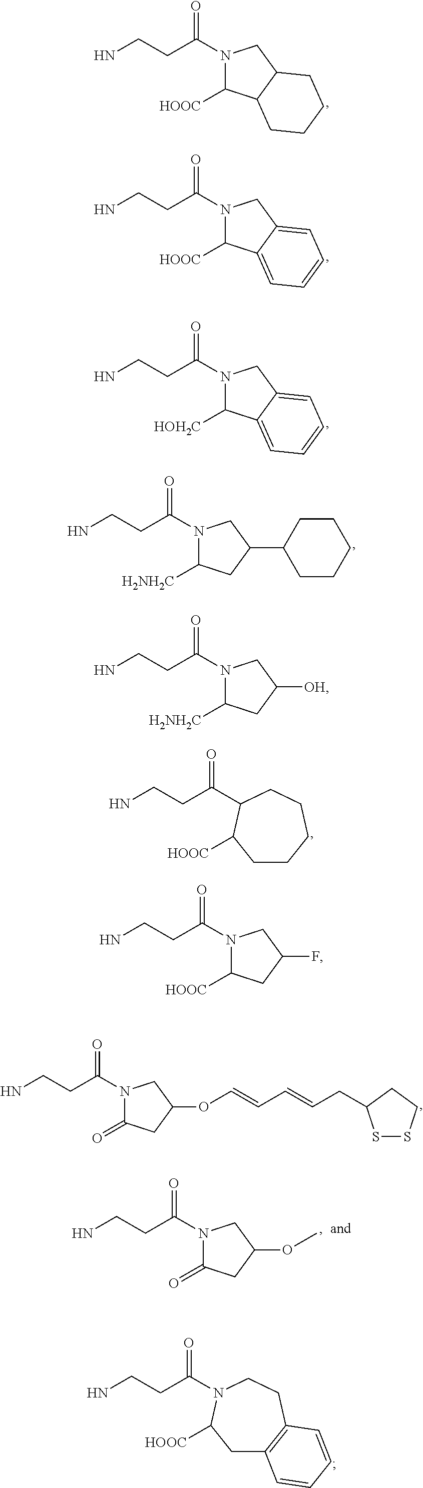 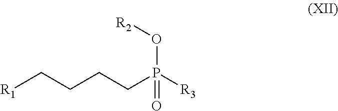
wherein:
R₁ is selected from the group consisting of: 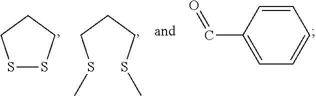
R₂ is selected from the group consisting of:
H, 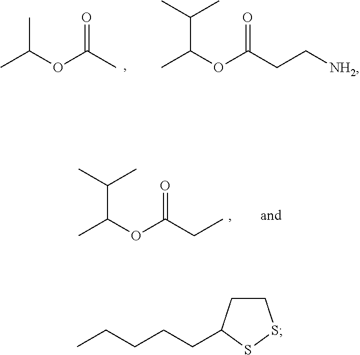
and
R₃ is selected from the group consisting of: 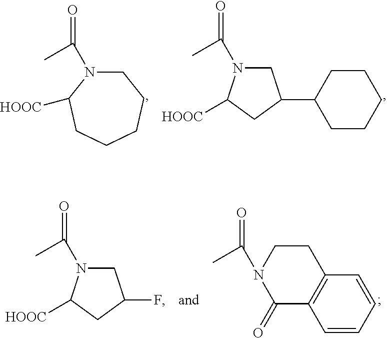 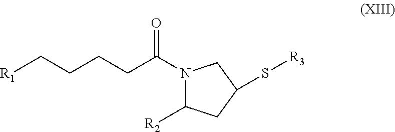
wherein:
R₁ is selected from the group consisting of: 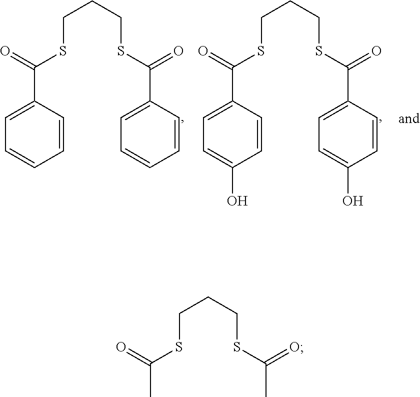
R₂ is selected from the group consisting of:
COOH, 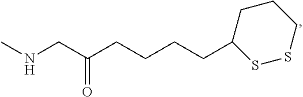
and CH₂OCH₃; and
R₃ is selected from the group consisting of: H, CH₃, and COCH₃; 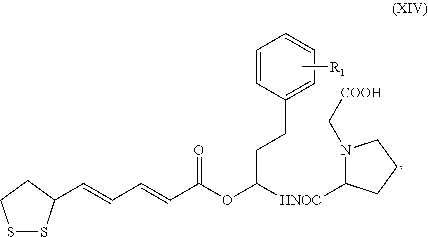
wherein R₁ is selected from the group consisting of OH, NO₂, NH₂, and OCH₃; 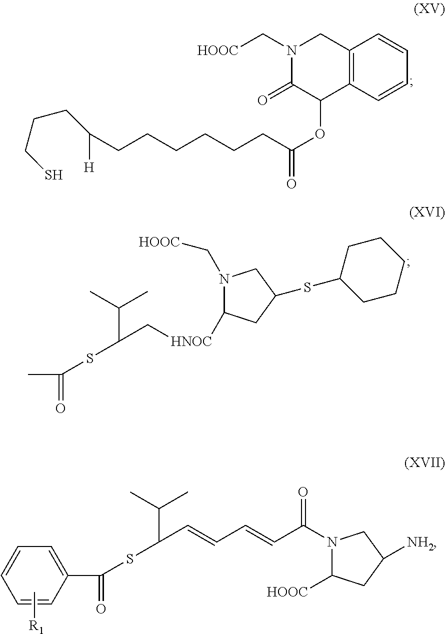
wherein R₁ is selected from the group consisting of OH, NO₂, OCH₃, and NH₂; and 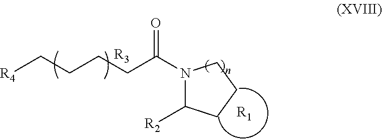
wherein R₁ is an optional ring that can be selected from the group consisting of a cyclopentyl ring, a cyclohexyl ring, a phenyl ring, and a substituted phenyl ring where the substituents on the phenyl ring are selected from the group consisting of OH, OCH₃, NH₂, NO₂, COOH;
wherein R₂ is selected from the group consisting of a free carboxyl, methyl or ethyl ester, a primary alcohol, and a primary amine group;
wherein n is an integer from 1 to 3, and the nitrogen-containing ring is a substituted or unsubstituted ring selected from the group consisting of a pyrrolidine ring, a piperidine ring, and a saturated azepine ring;
wherein R₃ is an alkyl group that includes from 1 to 3 carbon atoms, and which can include one or more conjugated or unconjugated double bonds; and
wherein R₄ is a substituted or unsubstituted 5- or 6-membered disulphide moiety or a 5- or 6-membered dithiol moiety selected from the group consisting of

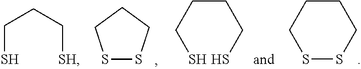

40. A method of treating stroke comprising administering to a patient in need thereof an effective amount of a compound selected from the group consisting of the following Formulas (I) and (X)-(XVIII), or pharmaceutically acceptable salts or solvates thereof: 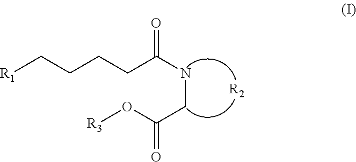
wherein:
R₁ is selected from the group consisting of 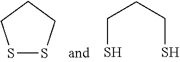
R₂ completes a 5- or 6-membered non-aromatic heterocyclic ring structure; and
R₃ is selected from the group consisting of CH₃ and H; 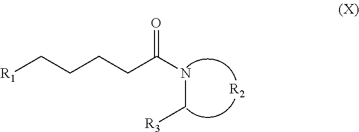
wherein:
R₁ is selected from the group consisting of: 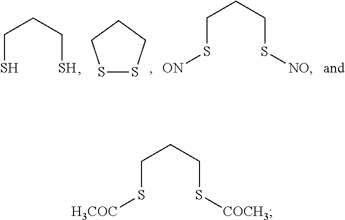
R₂ completes a five- or six-membered non-aromatic heterocyclic ring structure optionally containing a hydroxyl substituent, or an eight- or ten-membered bicyclic aromatic or non-aromatic heterocyclic ring structure; and
R₃ is selected from the group consisting of:
COOH, CH₂OH, 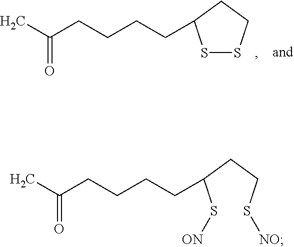
or a pharmaceutically acceptable salt or solvate thereof; 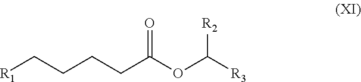
wherein:
R₁ is selected from the group consisting of: 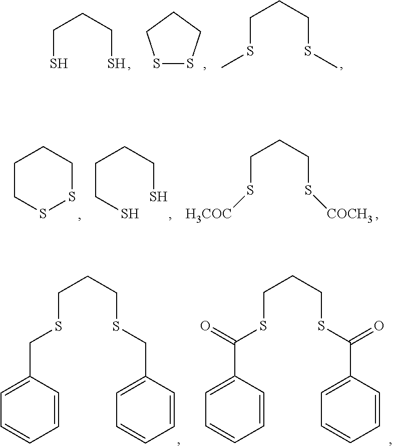
and;
R₂ is selected from the group consisting of:
(CH₂)₂CH₃, (CH₂)₃NH₂, (CH₂)₃OH, (CH₂)₄COOH, 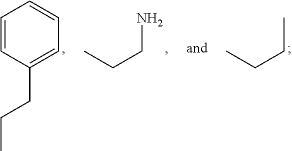
and
R₃ is selected from the group consisting of: 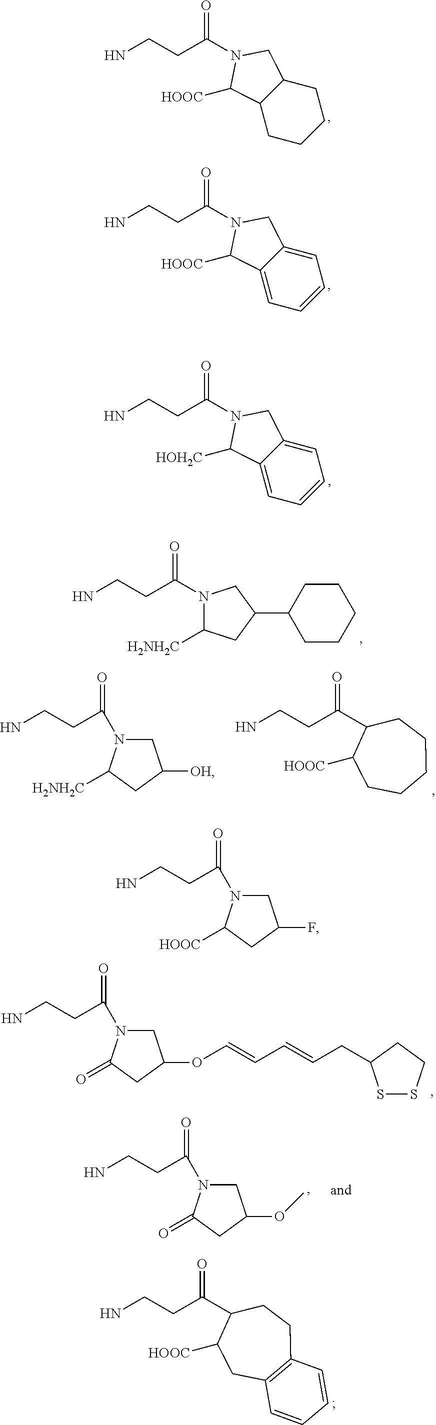 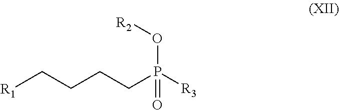
wherein:
R₁ is selected from the group consisting of: 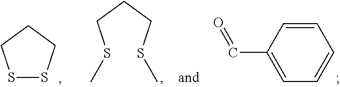
R₂ is selected from the group consisting of: 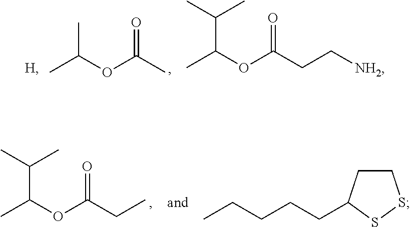
and
R₃ is selected from the group consisting of: 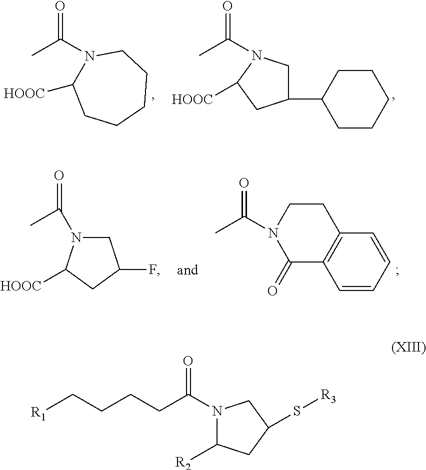
wherein:
R₁ is selected from the group consisting of: 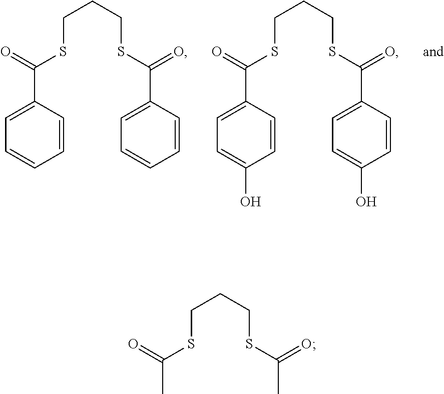
R₂ is selected from the group consisting of:
COOH, 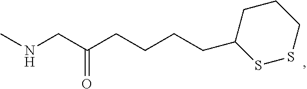
and CH₂OCH₃; and
R₃ is selected from the group consisting of: H, CH₃, and COCH₃; 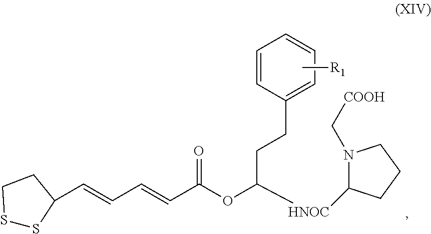
wherein R₁ is selected from the group consisting of OH, NO₂, NH₂, and OCH₃; 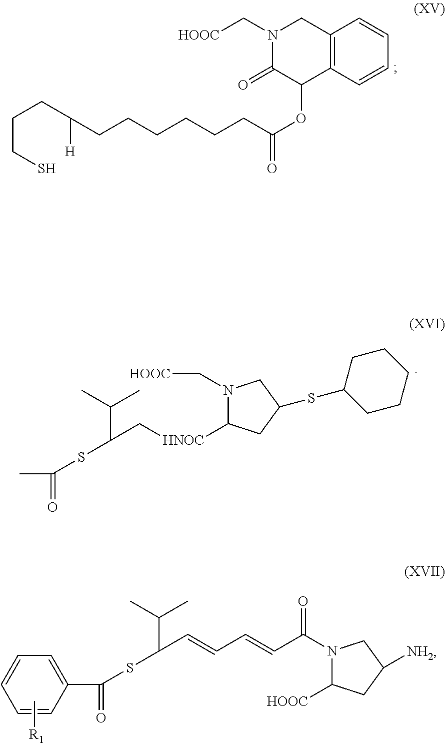
wherein R₁ is selected from the group consisting of OH, NO₂, OCH₃, and NH₂; and 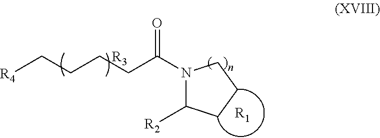
wherein R₁ is an optional ring that can be selected from the group consisting of a cyclopentyl ring, a cyclohexyl ring, a phenyl ring, and a substituted phenyl ring where the substituents on the phenyl ring are selected from the group consisting of OH, OCH₃, NH₂, NO₂, COOH, and CONHR, where R is methyl or ethyl;
wherein R₂ is selected from the group consisting of a free carboxyl, methyl or ethyl ester, a primary alcohol, and a primary amine group;
wherein n is an integer from 1 to 3, and the nitrogen-containing ring is a substituted or unsubstituted ring selected from the group consisting of a pyrrolidine ring, a piperidine ring, and a saturated azepine ring;
wherein R₃ is an alkyl group that includes from 1 to 3 carbon atoms, and which can include one or more conjugated or unconjugated double bonds; and
wherein R₄ is a substituted or unsubstituted 5- or 6-membered disulphide moiety or a 5- or 6-membered dithiol moiety selected from the group consisting of [chemical structure]
